(19) United States
(12) Patent Application Publication
(10) Pub. No.: US 2026/0027375 A1
(43) Pub. Date: Jan. 29, 2026

(54) SYSTEMS AND METHODS FOR ELECTROCONVULSIVE THERAPY

(71) Applicants: Christopher Abbott, Albuquerque, MN (US); Zhi-De Deng, Bethesda, MD (US); Joel Upston, Albuquerque, MN (US); Thomas Jones, Albuquerque, MN (US); Abhisheck Datta, Watchung, NJ (US)

(72) Inventors: Christopher Abbott, Albuquerque, MN (US); Zhi-De Deng, Bethesda, MD (US); Joel Upston, Albuquerque, MN (US); Thomas Jones, Albuquerque, MN (US); Abhisheck Datta, Watchung, NJ (US)

(21) Appl. No.: 19/145,154

(22) PCT Filed: Jan. 4, 2024
(86) PCT No.: PCT/US2024/010363

§ 371 Date: Jul. 1, 2025

Related U.S. Application Data
(60) Provisional application No. 63/437,017, filed on Jan. 4, 2023.
(60) Provisional application No. 63/516,371, filed on Jul. 28, 2023.

(51) Int. Cl. A61N 1/38

(52) U.S. Cl. CPC A61N 1/38

(57) ABSTRACT

The present disclosure relates to methods of electroconvulsive therapy (ECT) for a patient's brain based on determining an optimal individualized current amplitude for a patient by (1) dividing an optimal E-field strength (optimal Ebrain) by a baseline E-field strength (baseline Ebrain) of the patient's brain, or (2) performing an initial treatment to determine a patient's amplitude titrated seizure threshold (STa), followed by use of a multiplier value multiplied by the STa for an individualized amplitude for subsequent treatments. Methods of ECT using optimal individualized current amplitude provide an antidepressant effect and reduce adverse cognitive effects on the patient's brain.

### CROSS-REFERENCE TO RELATED APPLICATION

This application claims the priority of U.S. provisional application Ser. No. 63/437,017, filed Jan. 4, 2023, and U.S. provisional application Ser. No. 63/516,371, filed Jul. 28, 2023, the disclosure of each application is incorporated herein by reference in their entirety.

### FEDERAL FUNDING

This invention was made with government support under MH125126, MH128692, and MH111826 from the National Institute of Mental Health (NIMH). The government has certain rights in the invention.

### BACKGROUND OF THE INVENTION

Major depression (MDD) ranks second of all diseases in global impact and produces high morbidity and mortality rates. Among FDA-cleared antidepressant treatments (e.g., pharmacotherapy, transcranial magnetic stimulation, ketamine) for MDD and treatment resistant depression (TRD), electroconvulsive therapy (ECT) produces the highest response and remission rates.

ECT uses electric current to induce seizure activity under general anesthesia. While ECT is highly efficacious, cognitive adverse effects limits use. ECT produces adverse cognitive effects (incidence rate=74%) that can persist up to 6 months, and attenuate clinical improvement, which limits its use. Key adverse cognitive effects include amnesia, executive dysfunction, and verbal dysfluency. Unfortunately, the mechanisms underlying antidepressant and cognitive adverse effects, which could advance and guide more effective ECT practice, remain unknown.

Currently, most ECT clinicians implement a trial-and-error approach to parameter selection, initially favoring reduced cognitive risk (right unilateral (RUL) electrode placement and ultrabrief pulse width) before advancing to the more efficacious but increased cognitive risk parameter selections (BT and brief pulse width) (American Psychiatric Association, 2001). This trial-and-error approach may still expose patients to increased cognitive risk with RUL (i.e., patient has high E_{brain}) and may miss the optimal dose for antidepressant response without cognitive risk.

### BRIEF DESCRIPTION OF THE DRAWINGS

FIG. 1 is a schematic illustrating two types of electrode placement used in ECT, right unilateral (RUL) and bitemporal (BT) and shows stimulus wave form.

FIG. 2 is an illustration of electric field (E-field) modeling that creates a subject-specific, anatomically realistic volume conductor model.

FIG. 3 is an illustration of the sufficiency of ECT dosing and their clinical outcomes.

FIG. 4 is a graph of a ROC curve analysis comparing right hippocampal E-field and cognitive outcome (ΔLetter Fluency>−3).

FIG. 5 is a graph of a ROC curve analysis showing that Eₕᵢₚₚₒ is related to E_{brain}.

FIG. 6 is a graph showing higher E_{brain} was related to cognitive impairment.

FIG. 7 is a graph showing the antidepressant effect (blue line) and cognitive impairment (red line) associated with excessing ECT dosing and optimal ECT dosing.

FIG. 8 is a flow chart showing subject (e.g., patient) flow and attrition based on pre- (V1), mid- (V2), and post-ECT (V3) time points.

FIGS. 9A-9C are graphs depicting the range of amplitude-determined seizure-titration (STₐ) and E_{brain}, the median STₐ (mA), and the median E_{brain}. FIG. 9A is a graph showing that E_{brain} was associated with amplitude-determined seizure (STₐ, eta-squared=0.26). Older subjects had higher STₐ (color bar, eta-squared=0.21). FIG. 9B is a graph showing STₐ and treatment number interaction was associated with right hippocampal volume change (eta-squared=0.24). Lower STₐ had an earlier right hippocampal volume increase that plateaued with higher treatment number. FIG. 9C is a conceptual figure illustrating a method of amplitude-determined seizure titration. The first treatment determined an amplitude seizure threshold (STa). Subsequent treatments were completed at 800 mA. The resultant amplitude ratio 800 mA/STa determines the relative dose of ECT. Low STa followed by 800 mA will result in a “high” dose and cognitive impairment. High STa followed by 800 mA will result in a “low” dose that is insufficient for antidepressant response.

FIGS. 10A-10B are graphs showing antidepressant outcomes as it relates to STₐ and right hippocampal volume. FIG. 10A is a graph showing higher STₐ was associated with reduced change in depression severity at the mid-ECT assessment (eta-squared=0.20) and differentiated bitemporal electrode placement switch. The dashed line is the cut-point (328 mA) associated with bitemporal electrode placement switch (area under the ROC curve: 0.85). FIG. 10B is a graph showing right hippocampal volume change was associated with antidepressant outcomes (eta-squared=0.20) and differentiated RUL response criteria (3.1% volume change, dashed red vertical line).

FIG. 11 shows a brain analysis comparing regional electric field strength and category fluency outcomes which revealed widespread right hemisphere associations. Increased E_{brain}/I was associated with impaired DKEFS Category Fluency performance (eta-squared=0.29). Whole brain analysis revealed widespread right hemisphere relationships with increased regional E-field strength and impaired cognitive performance (54 of 166 regions with p_{FDR}<0.05).

FIG. 12 is a flow chart of an exemplary amplitude-determined seizure-titration method.

### DETAILED DESCRIPTION

Electroconvulsive therapy (ECT) is one of the most effective treatments for adults with major depressive disorder (MDD) and is also used to treat a variety of other mental health disorders. The antidepressant effect is seen relatively quickly and may last up to a few years. However, some patients fail to remit despite an adequate course, and even when effective, ECT can produce cognitive impairment including amnesia, executive dysfunction, and verbal dysfluency. While some (right unilateral, RUL) relative to other (bitemporal, BT) ECT modalities of extracranial electrode placement result in less cognitive impairment, they still produce moderate to large (Cohen's 19d=−0.53 to −0.83) adverse cognitive effects.

Conventional ECT methods include a trial-and-error approach of ECT parameter selection. The elusive underlying mechanisms of ECT limit the ability to dissociate antidepressant benefit from cognitive side effects. Previous single- and multi-site (Global ECT MRI Collaboration (GEMRIC)) ECT-imaging studies of mechanisms of antidepressant and cognitive outcomes have been limited by: 1) non-standardized ECT parameters, 2) insufficient sample sizes, 3) limited cognitive phenotyping, 4) non-harmonized imaging protocols, 5) and limited integration of ECT dosing via electroencephalography (EEG) and electric field (E-field) modeling. Thus, there is a gap in the knowledge that is needed to optimize ECT.

Fixed-amplitude ECT results in variable E-field strength due to individual conductivity and anatomical differences, which differentially impacts antidepressant (higher amplitudes better) and cognitive (lower amplitudes better) outcomes. Specifically, increased E-field strength is associated with increased hippocampal neuroplasticity, which is related to improved antidepressant outcomes. In contrast, increased E-field strength is also associated with worse cognitive outcomes as measured by the Delis Kaplan Executive Function System Verbal Fluency (DKEFS-VF) Test.

Traditional ECT dosing utilizes a fixed amplitude with the initial treatment devoted to seizure titration. The pulse number (combination of increasing pulse train duration and/or frequency) determines the seizure threshold determined as the minimum number of pulse-pairs or charge necessary to induce a seizure. Subsequent treatments are then completed with a multiplier (6×seizure threshold for right unilateral electrode placement) for antidepressant efficacy.

ECT is indicated in patients with treatment-resistant depression or severe major depression that impairs activities of daily living. Treatment-resistant depression refers to depression that is unresponsive to multiple antidepressant medication trials. ECT can also be used to treat other neuropsychiatric disorders including suicidality, severe psychosis, food refusal secondary to depression, schizophrenia, schizoaffective disorder, and catatonia. Bipolar depressive and manic patients can also receive treatment with ECT. ECT may have a safer profile than antidepressants or antipsychotics in debilitated, elderly, pregnant and breastfeeding patients. Suicidal ideation is rapidly relieved by ECT, and complete resolution has been seen in 38% of patients after one week, 61% of patients after two weeks and in 81% of patients with completion of ECT. ECT is also recommended for patients that have exhibited a favorable response to ECT previously.

ECT dosing can be divided into three categories for the ECT responder: insufficient (no antidepressant response, no cognitive impairment), optimal (antidepressant response, no cognitive impairment), or excessive (antidepressant response, cognitive impairment). Most ECT clinicians will use a trial-and-error approach to dosing that starts with parameters associated with less antidepressant efficacy and less cognitive risk and advances to parameters more associated with antidepressant efficacy and increased cognitive risk as a contingency.

Consequences of ECT-cognitive impairment include reluctance to consider initiation of the procedure, hesitancy to restart ECT in the context of relapse, perpetuation of ECT's stigma, and prolonged period of functional impairment. Importantly, ECT-cognitive impairment is unnecessary for antidepressant response and is a modifiable side effect. Efforts to improve ECT's risk/benefit ratio have often compromised antidepressant efficacy for cognitive safety.

Disclosed herein are methods of ECT comprising determining an optimal individualized current amplitude for each patient that elicits the desired antidepressant effect without adverse cognitive side effects. The optimal individualized current amplitude provides a personalized approach to ECT as an alternative to fixed current amplitude dosing that can result in variability in electric field (E-field) dosing. The fixed current amplitude approach fails to consider patients' individual anatomic variability, such as skull thickness, head diameter, and brain volume, which influences the E-field produced by ECT dosing. Individualized current amplitude can also eliminate the trial-and-error methods of ECT parameter selection thus reducing the overall number of treatments of the ECT series and bringing relief to the patient sooner.

### Individualized Current Amplitude by Determining Patient's Baseline E_{brain}

The methods of determining the optimal individualized current amplitude disclosed herein comprise determining a patient's baseline E-field strength (baseline E_{brain}) and dividing an optimal E-field strength (optimal E_{brain}) by the patient's baseline E_{brain} to produce the optimal individualized current amplitude. The patient's baseline E_{brain} can be determined by finite element modeling using structural MRI of the patient's brain with a fixed placement of extracranial electrodes. The baseline E_{brain} comprises at least a 90th percentile of E-field magnitudes from all voxels in the patient's brain. The patient's baseline E_{brain} can typically range from approximately 0.1 Volts/meter per milliampere to approximately 0.2 Volts/meter per milliampere. However, any given patient will have a baseline E_{brain} based on their own anatomic variability which may vary widely from person to person, their age, their gender, brain injuries, brain disorders, and other factors.

The optimal E_{brain} is an E-field strength sufficient to induce an antidepressant effect without cognitive impairment as determined by the clinical trials 1 and 2 described below in Examples 1 and 2. The antidepressant effect can include an improvement in the patient's score on a depression rating scale, such as Inventory of Depressive Symptomatology or Hamilton Depression Rating Scale-24 item (HDRS24). Cognitive impairment can include one or more of amnesia, executive dysfunction, or verbal dysfluency. The optimal E_{brain} was experimentally determined to be approximately 110 Volts/meter to approximately 120 Volts/meter.

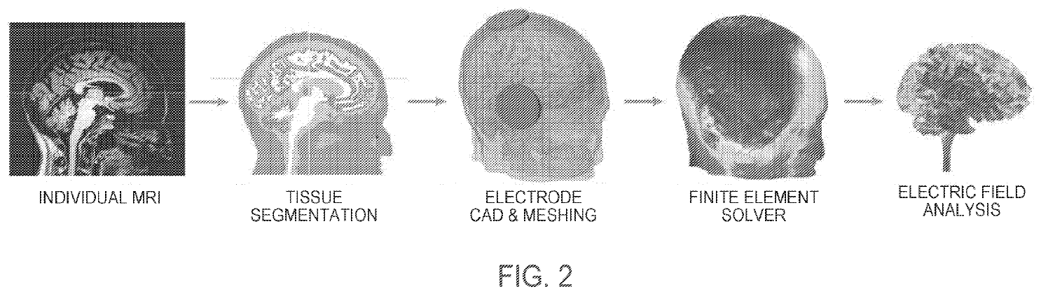

Therapeutic components of ECT include the electric field and induced seizure. Individual electric field (E-field) models can be created with finite element modeling using structural MRI (sMRI). Realistic head model development for electric stimulation includes segmentation of the brain, electrode placement on the patient's scalp, tessellation of volume into a mesh, and solving for the E-fields with the finite element method (determining the patient's baseline E_{brain}) (FIG. 2). With respect to ECT parameters, the electrode placement on the patient's scalp determines an E-field geometric shape, and current amplitude determines the E-field magnitude within the E-field shape. Research has demonstrated that the E-field is influenced by individual anatomic variability (skull thickness, head diameter, brain volume). Therefore, the individual's baseline E-field is calculated per milliampere of current. Tissue boundary effects could bias the absolute maximum E-field values. Therefore, percentages of maximal values (90 or 95%) are used to summarize an individual's E-field. Baseline E-field strength (E_{brain}) is the 90th percentile of E-field magnitudes from all voxels in the brain (typical range between 0.1 and 0.2 Volts/meter per milliampere of current). The E-field strength associated with an applied amplitude can be determined by multiplying the baseline E_{brain} by the current strength. Applying the baseline E_{brain} range of 0.1 to 0.2 Volts/meter per milliampere of current by 800 milliamperes creates a range of 80 to 160 Volts/meter. The E_{brain} can also be segmented to specific anatomic regions, such as the hippocampus (Eₕᵢₚₚₒ). E_{brain} and Eₕᵢₚₚₒ are strongly correlated (R2=0.61).

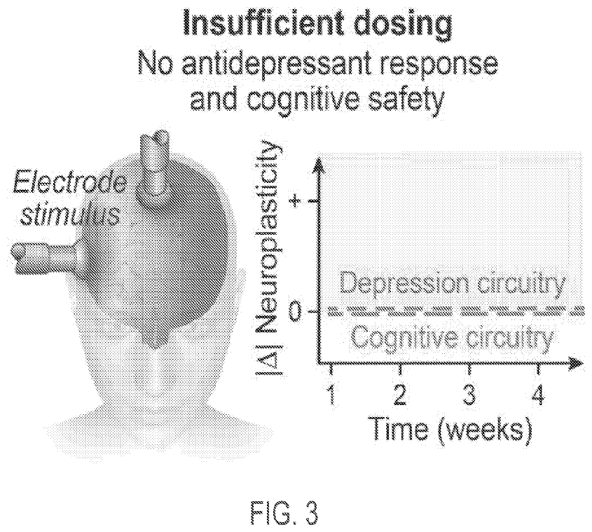

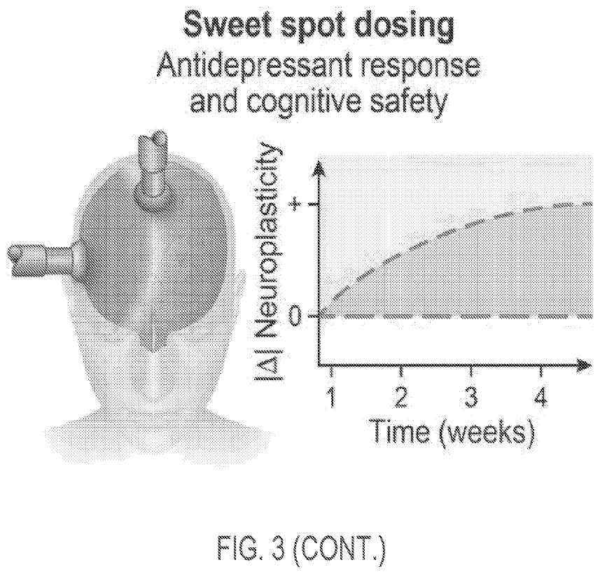

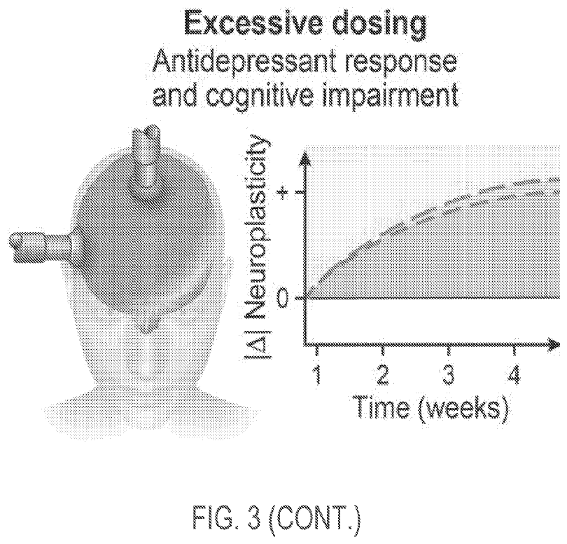

With respect to electric field modeling, ECT dosing can be divided into three categories for the ECT responder: insufficient (no antidepressant response, no cognitive impairment), optimal (antidepressant response, no cognitive impairment), or excessive (antidepressant response, cognitive impairment) (FIG. 3). Most ECT clinicians will use a trial-and-error approach to dosing that starts parameters associate with less antidepressant efficacy and less cognitive risk and advance to the more parameters associated with more antidepressant efficacy and increased cognitive risk as a contingency.

Fixed amplitude produces variable electric fields secondary to individual neuroanatomic differences such as differences in head diameter, skull thickness, or brain size. Adjustments to pulse width, pulse train duration, and frequency do not improve the efficacy of insufficient dosing or mitigate the cognitive risk of excessive dosing.

### Individualized Current Amplitude by Seizure Titration

The methods of determining the optimal individualized current amplitude disclosed herein comprise determining a patient's amplitude-determined seizure threshold during a first treatment of an ECT series with right unilateral (RUL) electrode placement. An amplitude-determined seizure titration procedure starts with applying a current at a low amplitude (i.e., 100 milliamperes (mA)) with incremental increases in amplitude (e.g., a 1 mA step size, a 25 mA step size, a 50 mA step size, or a 75 mA step size) until the patient has a seizure. The other ECT parameters (pulse width, frequency, and pulse train duration) will be fixed during the amplitude-determined seizure titration. A range of amplitudes at which seizure activity can occurs is from approximately 100 mA to approximately 700 mA. Despite this wide amplitude range, the E-field strength will have reduced variability. Subsequent treatments will be completed with an amplitude multiplier value such as approximately 2 to approximately 3 times the amplitude-determined seizure threshold or STₐ) for antidepressant efficacy. For example, the multiplier value can be 2.5 times the STₐ determined for a patient. Applying the STₐ multiplied by and multiplier value will ensure an antidepressant effect with reduced cognitive impairment as determined by the clinical trial described below in Example 4. The antidepressant effect can include an improvement in the patient's score on a depression rating scale, such as Inventory of Depressive Symptomatology or Hamilton Depression Rating Scale-24 item (HDRS24). Cognitive impairment can include one or more of amnesia, executive dysfunction, or verbal dysfluency.

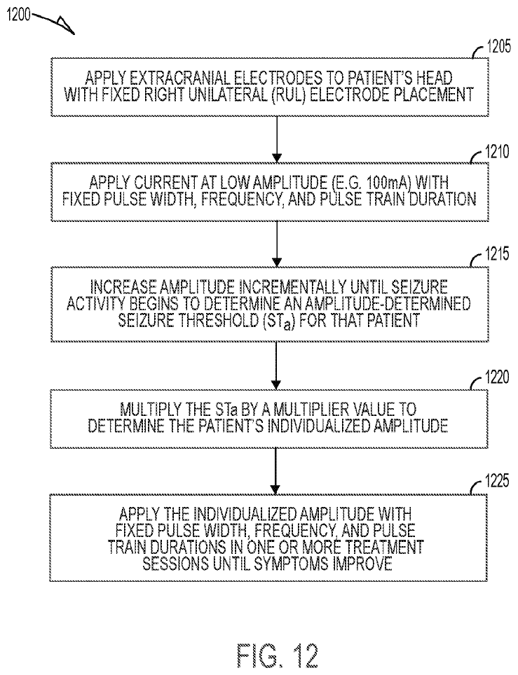

An exemplary amplitude-determined seizure-titration method 1200 is shown in FIG. 12. A first treatment of an ECT series includes applying extracranial electrodes to a patient's head with right unilateral (RUL) electrode placement (1205). A low amplitude current is applied with a fixed pulse width, frequency, and pulse train duration (1210). The amplitude is increased incrementally until seizure activity begins. The amplitude at which seizure activity begins is an amplitude-determined seizure threshold (STₐ) for the patient (1215). The STₐ determined for the patient is multiplied by a multiplier value to determine the patient's individualized amplitude (1220). The individualized amplitude is applied with a fixed pulse width, frequency, and pulse train durations in one or more subsequent treatment sessions until the patient's neuropsychiatric disorder symptoms improve (1225).

### Methods of Use

The methods described herein can be administered to treat human patients in need of such treatment, or who may develop a need for such treatment. For example, the methods can reduce the severity of major depressive disorder another other neuropsychiatric disorders while reducing or preventing adverse cognitive effects of the treatment.

### EXAMPLES

### Example 1: Methods for Determining Individualized Current Amplitude by Determining Patient's Baseline E_{brain}

### A. Clinical Assessment

The Mini International Neuropsychiatric Interview (MINI-7.0 for DMS-5) is a structured psychiatric interview that will confirm MDD presence and any exclusion psychiatric disorders. To measure depression symptoms and severity, primarily we will use the Clinician-Rated Inventory of Depressive Symptomatology (IDS-C) and secondarily the Self-Report IDS (IDS-SR)198-202. Psychotic symptoms will be measured with the Psychotic Depression Assessment Scale (PDAS). Any treatment emergent manic symptoms will be measured with the Young Mania Rating Scale (YMRS)204. Psychotropic medication use/treatment resistance will be measured with the Antidepressant Treatment History Form-Short-Form.

### B. Cognitive Assessment.

The cognitive battery is selected based on the need for 1) assessing specific ECT-induced cognitive adverse effects (e.g., amnesia, verbal dysfluency), 2) assessing select cognitive domains (e.g., declarative memory, language, cognitive control) based on the NIMH RDoC framework, 3) including estimated premorbid intelligence in statistical models, and 4) psychometrically sound cognitive measures with normative data and alternate forms to minimize confounding demographic and practice effects.

The cognitive assessment can comprise the following measures: 1) Montreal Cognitive Assessment (MoCA), 2) Electroconvulsive Therapy Cognitive Assessment (ECCA), 3) Measurement of Everyday Cognition (ECog), 4) Test of Premorbid Function (TOPF), 5) California Verbal Learning Test—3rd Edition (CVLT-3), 6) Autobiographical Memory Test (AMT), 7) two tests from the Delis-Kaplan Executive Function System (D-KEFS; Verbal Fluency, Color-Word Interference), and 8) NIH PROMIS Cognitive Abilities and Concerns Questionnaires. The MoCA, a global cognitive function measure, and the ECog, an informant-based measure sensitive to gross neurocognitive abnormalities in patients with MDD, will be used for screening. The ECCA was created specifically to assess neurocognitive effects of ECT. The TOPF estimates premorbid intelligence. The CVLT-3 is a ‘gold standard’ measure of verbal learning and memory. The AMT measures autobiographical memory recall and specificity. The D-KEFS tests measure verbal fluency and executive functions. The two NIH PROMIS measures provide self-report assessment of positive and negative aspects of cognitive function.

### C. MRI Acquisition and Pre-Processing

3T-Siemens scanner acquired T1 data with the following parameters: Repetition time (TR)=2530 milliseconds (ms), echo time (TE)=1.64, 3.5, 5.36, 7.22, 9.08 ms, Inversion time (TI)=1200 ms, flip angle=7.0°, slices=192, field of view=256, matrix 256×256, voxel size=1.0×1.0×1.0 millimeter (mm) and total acquisition time 6:03 (minutes:seconds). T2 data was collected with the following parameters: TR=2530 ms, TE=474 ms, flip angle=120.0°, slices=192, field of view=256, matrix 256×256, voxel size=1.0×1.0×1.0 mm and total acquisition time=5:09. FreeSurfer 6.0 segmented the cortical and subcortical anatomy with a longitudinal pipeline. This provides a robust and reliable estimation of the subcortical volumes and cortical thickness by creating an unbiased within-subject template image using inverse consistent registration. We processed all the time points separately with the default FreeSurfer workflow and created an unbiased template from all the time points for each subject. Once this template was created, parcellations and segmentation were carried out at each time point initialized with common information from the within-subject template. We identified the bilateral hippocampal volumes and calculated the percent changes in these regions relative to the pre-treatment volume.

### D. E-Field Modeling

We used the Simulation of Non-Invasive Brain Stimulation (SimNIBS) software for E-field modeling. SimNIBS creates a subject-specific, anatomically realistic volume conductor model. The T1- and T2-weighted scans are segmented into skin, bone, eyes, cerebral spinal fluid, ventricles, and grey and white matter with a combination of FMRIB Software Library (FSL) and Statistical Parametric Mapping 12 (SPM12) Computational Anatomy Toolbox. SimNIBS then turns this segmentation into a tetrahedral head mesh using Gmsh, a three-dimensional finite element (FE) mesh generator. Gmsh provide unique conductivity values for each tissue type: cerebrospinal fluid: (1.654 Siemens/meter (S/m)), vitreous bodies (0.50 S/m), scalp (0.465 S/m), gray matter (0.275 S/m), white matter (0.126 S/m), spongy bone (0.025 S/m), and compact bone (0.0008 S/m. ECT electrodes are added to the head mesh in either RUL or BT configuration and stimulated with the corresponding current. SimNIBS then uses a FE solver to calculate the voltages and electric fields that correspond to the stimulation throughout the head mesh.

We calculated the right and left hippocampal E-field strength based on the electrode placement (RUL or BT) and amplitude (600, 700, 800 mA) from the last treatment of the ECT series. Hippocampal E-field strength (Eₕᵢₚₚₒ) was calculated as the 95^{th} percentile of E-field magnitudes from all voxels in the hippocampus, serving as an estimate of the peak-induced field strength while avoiding the influence of tissue boundary effects that could bias the absolute maximum E-field values. To balance the focused approach on the hippocampus, we also calculated E_{brain}/I_{electrode}, where E_{brain} is the 90^{th} percentile of E-field magnitude in the whole brain and I_{electrode} is the stimulation current⁵¹. This ratio is the induced E-field in the brain per unit of stimulation current. This metric depends only on the electrode placement and individual head anatomy and is independent of waveform parameters including current amplitude, and thus reflects only the spatial properties of the induced E-field in the brain.

### E. ECT Dosing

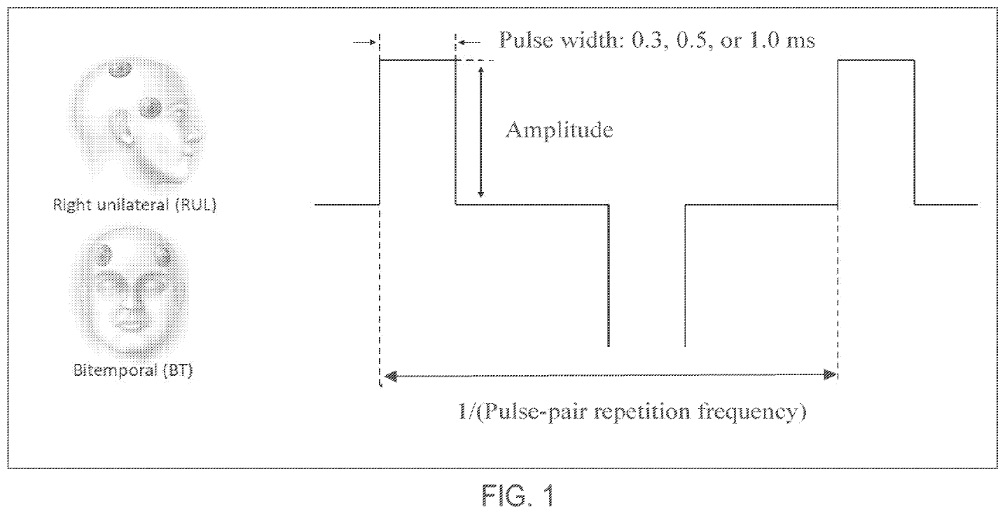

In some ECT methods, ECT parameter selection consists of electrode placement (right unilateral (RUL) or bitemporal (BT)), pulse width (0.3 to 1.0 milliseconds (ms)), frequency (20 to 120 hertz (hz)), pulse train duration (0.5 to 8 seconds (s)), and an optimal individualized current amplitude (typical range is approximately 600 milliamperes (mA) to approximately 1200 mA) to deliver a square pulse wave of alternating current (FIG. 1).

In some ECT methods, the ECT clinician selects electrode placement and pulse width prior to treatment. Demographic (age and sex) or seizure titration determine the frequency and pulse train for an individual patient. For demographic dosing, older subjects receive a higher pulse number. For seizure titration, the first session will determine the number of pulses that elicit a seizure with a method of limits. Subsequent sessions will use a multiplier of the total charge (proxy for pulse number) to determine the pulse number for subsequent supra-seizure threshold treatments (2×seizure threshold for BT, 6×seizure threshold for RUL). The first ECT session consists of dose titration with repeated sub-convulsive stimulations until a seizure is produced with increased pulse train duration and frequency (amplitude is fixed). Subsequent treatments are completed at six-times the seizure threshold charge with additional increases in pulse train duration and frequency (amplitude remains fixed). Both methods of dosing utilize a fixed amplitude of 800 or 900 mA dependent on the ECT device. Parameters associated with less cognitive risk include RUL electrode placement and ultrabrief pulse width (<0.5 ms). Parameters associated with more antidepressant efficacy include BT electrode placement and brief pulse width (>0.5 ms).

### F. Optimal Individualized Current Amplitude

Prior to the initiation of ECT, E-field modeling can determine in individual's baseline E_{brain}. The optimal individualized current amplitude can be determined from the calculation: optimal E-field (optimal E_{brain}) (V/m)/baseline E-field (baseline E_{brain}) (V/m per mA). For example, if an exemplary patient #1 has a baseline E_{brain} of 0.133 Volts/meter per milliampere. The individualized amplitude will be 900 milliamperes (120 Volts/meter/0.133 Volts/meter per milliampere). In another example, an exemplary patient #2 has a baseline E_{brain} of 0.2 Volts/meter per milliampere. The individualized amplitude will be 600 milliamperes (120 Volts/meter/0.2 Volts/meter per milliampere). The successful implementation of using an individualized current amplitude on a research participant is detailed below in Example 3.

### G. General ECT Procedures

ECT procedures comply with the APA ECT Practice Guidelines and the recent US FDA ECT Device reclassification guidance. ECT can be provided three times per week with a MECTA SIGMA ECT device (MECTA Corp., Tualatin, Oregon) or similar device.

A complete history and physical examination is done to expose any significant risk factors including cardiac ischemia or arrhythmia, heart failure, hypertension, tachycardia or intracranial pathology. History should also include the use of herbal medications, prescription medications including cardiac medications such as aspirin, statins, antihypertensive agents, and antianginal medications. Serum glucose levels require checking both preoperatively and in the recovery room, as ECT treatments can raise blood glucose levels. Although ECT appears to be safe in a patient with a defibrillator, detection mode should be turned off during the procedure and equipment for external defibrillation should be available at the patient's bedside. In pregnant patients, noninvasive fetal monitoring is the recommendation after 14 to 16 weeks and a nonstress test with a tocometer after 24 weeks.

ECT is commonly performed in a dedicated suite, a post-anesthesia care unit, or an ambulatory surgery site, most frequently on an outpatient basis. Patients with severe debilitation including substantial medical or psychiatric illness may start on an inpatient basis and transition to an outpatient basis as needed. Patients should be appropriately nil per os (NPO) for the procedure, which includes no light meal for six hours, no full-fat meal for eight hours, and no clear liquids for two hours before anesthesia.

Vital signs, including blood oxygen saturation, ECG and EEG activity are recorded continuously. EMG is recorded on the right foot to measure the motor component of seizure activity. A nerve stimulator is utilized to monitor succinylcholine, a depolarizing muscle relaxant used to reduce tonic-clonic contractions during the procedure. As an alternative to EMG, a blood pressure cuff is inflated on the patient's ankle to prevent succinylcholine from entering the foot, allowing a visual monitor of seizure activity with measurement of tonic-clonic contractions. Following intravenous induction, a bite block is placed to protect the patient's tongue and teeth. The beginning and termination of a cerebral seizure is monitored via EEG, recorded from right and left frontal and mastoid positions. Seizure induction is via two electrodes placed bitemporally or a right unilateral electrode; both of which allow electrical current to pass into the scalp.

ECT utilizes general anesthesia. Anesthetic induction medications used include barbiturates such as thiopental and methohexital and nonbarbiturate agents such as propofol and etomidate. Seizure-induced by ECT should last longer than 30 seconds. Methohexital is commonly used as the induction agent due to its quick onset, effectiveness, low cost, and minimal effect on seizure duration. Propofol and thiopental have been shown to reduce seizure duration. Etomidate has correlations with myoclonus and increased seizure duration.

### Example 2: Clinical Trials to Determine Optimal E-Field Strength (Optimal E_{brain})

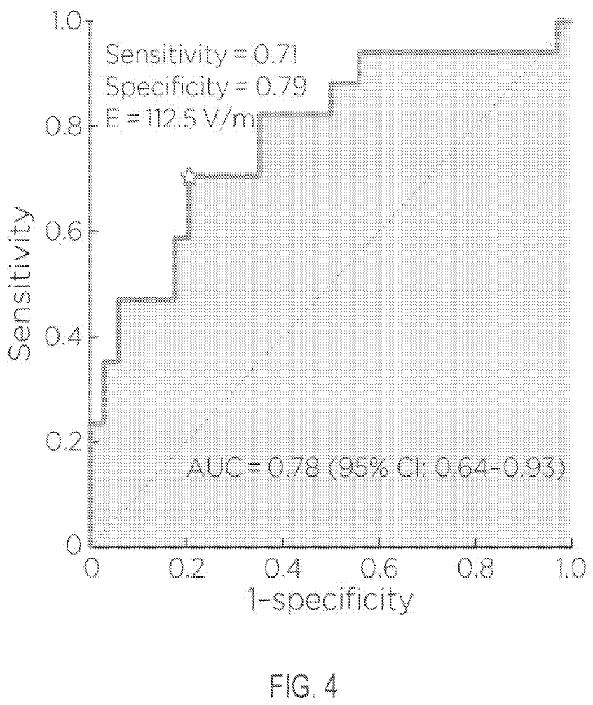

The amplitude associated with optimal dosing can be determined prior to the ECT series with pre-treatment structural MRI and E-field modeling. Our previous research from two different datasets has converged on an E-field strength associated with optimal clinical outcomes. The first dataset (MH111826, n=60) determined that higher amplitudes were associated with better antidepressant response, and lower amplitudes were associated with improved cognitive performance. Abbott C C, et al., Electroconvulsive Therapy Pulse Amplitude and Clinical Outcomes. Am J Geriatr Psychiatry. 2021 February; 29(2):166-178. doi: 10.1016/j.jagp.2020.06.008. Epub 2020 Jun. 17. PMID: 32651051; PMCID: PMC7744398. Increased E-fields (Eₕᵢₚₚₒ) were related to improved antidepressant outcomes only with a hippocampal volume change mediator. Deng Z D, et. al., Electroconvulsive therapy, electric field, neuroplasticity, and clinical outcomes. Mol Psychiatry. 2022 March; 27(3):1676-1682. doi: 10.1038/s41380-021-01380-y. Epub 2021 Dec. 1. PMID: 34853404; PMCID: PMC9095458. In contrast, the increased E-fields were strongly associated with impaired cognitive outcomes as measured by changes in verbal fluency. The optimal E-field (Eₕᵢₚₚₒ) was 112 Volts/meter to differentiate cognitive impairment outcomes (FIG. 4). Since the location of seizure generation is unknown, we changed focus from Eₕᵢₚₚₒ to E_{brain} and whole brain analyses.

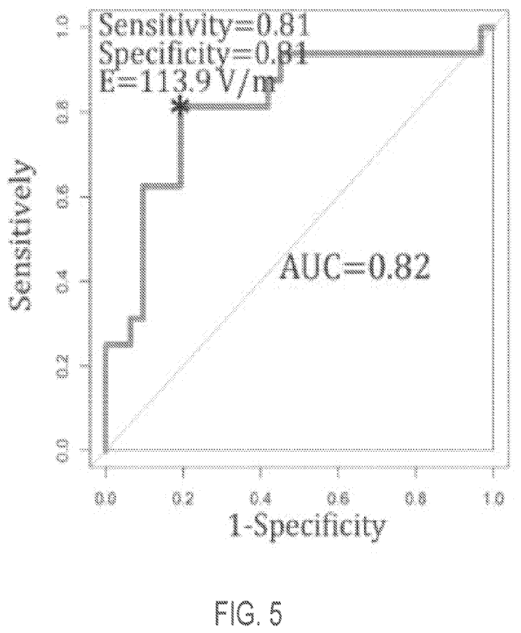

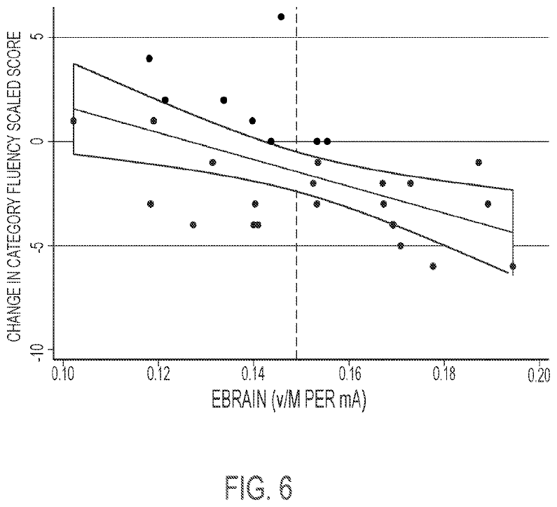

The next investigation with the same dataset used data-driven approach with both structural and functional (resting state fMRI) imaging. Qi S, et. al., Links between electroconvulsive therapy responsive and cognitive impairment multimodal brain networks in late-life major depressive disorder. BMC Med. 2022; 20(1):477. PubMed PMID: PMCID: PMC9733153. Despite the shift in imaging and analysis modalities, the results were consistent with our previous approach. E-field was modestly associated with antidepressant outcomes and strongly associated with cognitive outcomes. The optimal E-field (optimal E_{brain}) associated with cognitive outcomes was 114 Volts/meter (FIG. 5). A second dataset (MH125126, n=29) focused on methods to individualize amplitude. E-field was again modestly associated with antidepressant outcomes and strongly associated with cognitive outcomes. The optimal E_{brain} associated with cognitive outcomes was 119 Volts/meter (FIG. 6). The results from these different methodologies and datasets converge with the following results: 1) increased E-field strength is modestly associated with the improved antidepressant outcomes via neuroanatomic mediator (i.e., hippocampal volume change); 2) E-field strength is strongly association with cognitive outcomes; and 3) the cut-point with impaired cognitive performance is approximately 120 Volts/meter.

### Example 3: Clinical Implementation of Optimal Individualized Current Amplitude in a Research Subject

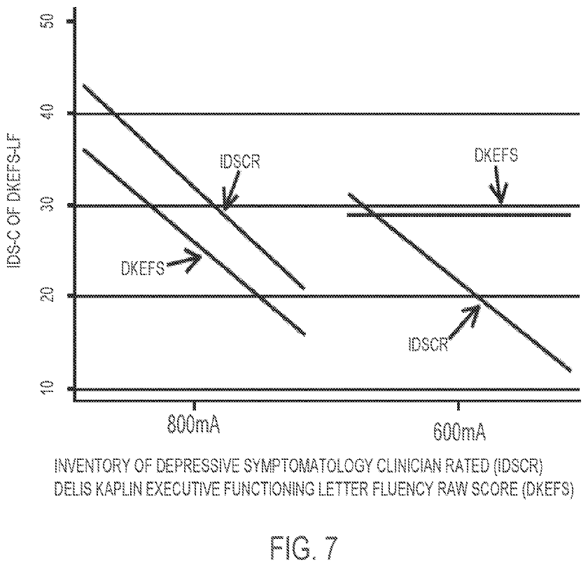

The optimal individualized current amplitude can be determined from the optimal E-field (optimal E_{brain}) (V/m)/baseline E-field (baseline E_{brain}) (V/m per mA). We have successfully implemented this method for individualized amplitude determination (FIG. 7). The subject was a research participant that developed ECT-mediated cognitive impairment with 800 milliampere with RUL extracranial electrode placement ECT. We calculated the patient's baseline E_{brain} as described above, which was 0.19 Volts/meter per milliampere. The ECT series was resumed with the optimal individualized current amplitude of 600 milliampere (120 Volts/meter/0.19 Volts/meter per milliampere=632 milliampere but rounding was necessary secondary to device limitation). The subject had antidepressant response and cognitive recovery with the remaining treatments.

The relationship between E-field strength and cognitive outcomes (optimal E_{brain}-numerator) relative to the patient's own baseline E-field (baseline E_{brain}-denominator) can therefore be used to determine the optimal individualized current amplitude for optimal dosing. Based on the clinical trial research disclosed herein, the optimal E_{brain} range for the desired cognitive outcomes is between 110-120 Volts/meter. Upcoming larger cohort clinical trials may further elucidate the optimal E_{brain} range for use in calculating the optimal individualized current amplitude.

### Example 3: Methods for Determining Individualized Current Amplitude by Seizure Titration

### A. Participants

The overall study design has been registered as a clinical trial (ClinicalTrials.gov Identifier: NCT04621786). The University of New Mexico Human Research Protections Office approved this investigation (20-601). All subjects provided written informed consent to the research protocol and study participation. Inclusion criteria consisted of the following: 1) major depressive disorder (MDD; with or without psychotic features) confirmed with two separate psychiatric evaluations (First et al., 2002); 2) clinical indications for ECT with right unilateral electrode placement including treatment resistance or a need for a rapid and definitive response (American Psychiatric Association, 2001); 3) right-handedness, and 4) age range between 50 and 80 years. Subjects remained on antidepressant treatment throughout the ECT series with antidepressant medication dose titrations permitted during the ECT series. Exclusion criteria consisted of the following: 1) Defined neurological or neurodegenerative disorder (e.g., traumatic brain injury, epilepsy, Alzheimer's disease); 2) other psychiatric conditions (e.g., schizophrenia, bipolar disorder) as the primary indication for ECT; 3) current drug or alcohol use disorder (except for nicotine); and 4) contraindications to MRI.

### B. Study Protocol

Subjects received their baseline imaging, clinical, and neuropsychological assessment 24 to 48 hours before the first ECT session (V1). Amplitude-determined seizure titration was completed during the first treatment with subsequent treatments completed with RUL and 800 mA. The ECT series continued thrice weekly until clinically determined endpoints (American Psychiatric Association, 2001). Subjects received their second assessment (V2) one day after the sixth ECT treatment and the final assessment (V3) within one week after the acute phase of treatment. The timing of the V2 assessment allows sufficient time to evaluate the effectiveness of RUL 800 mA and change to BT electrode placement if indicated to ensure that every patient receives an adequate ECT series. Each study visit included magnetic resonance imaging (MRI), clinical, and neuropsychological assessments. The anesthesiologist determined the appropriate dosage of general anesthetic (methohexital unless not available) and succinylcholine (depolarizing neuromuscular blocker). Anesthetic medications, electroencephalographic seizure duration, and maximum ictal heart rate were recorded for each treatment.

### C. Clinical Assessments

The clinician-rated 30-item Inventory of Depressive Symptomatology (IDS-C) measured depression severity (Rush et al., 1996). Each item is scored from 0-3 and summed for a total score between 0-84. The initial visit (V1) included the Maudsley Staging Method to measure antidepressant treatment resistance within the current depressive episode (Fekadu et al., 2009), ECT Appropriateness Scale to assess the indication for ECT (Kellner et al., 2012), Medical History form to gauge overall medical burden, and Edinburgh Handedness Inventory to define handedness (Oldfield, 1971). Additional characteristics of the current and past depressive episodes were also recorded during the initial visit: age of onset, age of first treatment, number of depressive episodes, and depressive episode duration.

### D. Neuropsychological Assessments

The Test of Premorbid Function (TOPF) estimated premorbid intellectual function for use as a covariate in cognitive analyses (Wechsler, 2009). The Montreal Cognitive Assessment (MoCA), a measure of global cognitive function that is sensitive to gross neurocognitive abnormalities, screened for preexisting cognitive impairment (Nasreddine et al., 2005; Rossetti et al., 2011). The Delis Kaplan Executive Function System (DKEFS) included Verbal Fluency (Letter and Category), Color-Word Interference (processing speed, inhibition, initiation, and cognitive flexibility), and the Tower Test (planning and problem-solving) (Delis et al., 2001; Delis et al., 2004; Latzman and Markon, 2010; Mitchell and Miller, 2008; Yochim et al., 2007). California Verbal Learning Test—3^{rd} Edition (CVLT-3) measured verbal learning and memory (Delis et al., 2017). The Dot Counting Test measured performance validity (Boone et al., 2002). The Digit Span subtest from the Wechsler Adult Intelligence Scale—4^{th} Edition (W AIS-IV) measured working memory (Wechsler, 2008). Our primary cognitive outcome was the DKEFS Verbal Fluency test (letter and category fluency) based on sensitivity to detect cognitive impairment with RUL (Abbott et al., 2021).

### E. MRI Acquisition

T1 data was collected with the following parameters: Repetition time (TR)=2530 milliseconds (ms), echo time (TE)=1.64, 3.5, 5.36, 7.22, 9.08 ms, Inversion time (TI)=1200 ms, flip angle=7.0°, slices=192, field of view=256, matrix 256×256, voxel size=1.0×1.0×1.0 millimeter (mm) and total acquisition time 6:03 (minutes:seconds). T2 data was collected with the following parameters: TR=2530 ms, TE=474 ms, flip angle=120.0°, slices=192, field of view=256, matrix 256×256, voxel size=1.0×1.0×1.0 mm and total acquisition time=5:09.

### F. Amplitude-Determined Seizure Threshold

Subjects received amplitude-determined seizure titration during the first treatment with RUL electrode placement (d'Elia, 1970). The United States Food and Drug Administration (US FDA) approved the Soterix Medical 4X1 High Definition ECT Multi-channel Stimulation Interface for use in this investigation (Investigation Device Exemption: G200123). For the purposes of this investigation, we only used the amplitude reducer function of the interface. Subjects received stimulations starting with the lowest setting ˜100 mA with ˜70 mA increases at 30 second intervals until seizure activity was initiated (STa). The delivered current was verified with an oscilloscope (RIGOL DS1074Z). EEG and right lower extremity motor activity confirmed seizure activity. Pulse width (1.0 milliseconds (ms)), pulse train duration (8 s), and frequency (20 hertz (Hz), 160 pulse pairs) were fixed for amplitude titration.

### G. Electroconvulsive Therapy

Pulse width (1.0 ms), pulse train duration (8 s), and frequency (20 Hz, 160 pulse pairs) were fixed for the remaining 800 mA RUL treatments. By fixing the temporal stimulus parameters, the only difference between amplitude determined seizure titration and subsequent treatments was amplitude. Relatively low frequency (20 Hz) was used since it induces seizures more efficiently. Brief pulses (1.0 ms) maximize antidepressant efficacy of RUL ECT. If the RUL 800 mA treatments failed to demonstrate antidepressant improvement at V2 (<25% reduction from baseline IDS-C30 total score), subjects would then receive BT electrode placement, fixed 800 mA amplitude, 1.0 ms pulse width, and traditional fixed amplitude seizure titration based on step-wise increases in pulse train duration and frequency. Subsequent BT treatments were then delivered at two times the charge for the remainder of the ECT series.

### H. FreeSurfer Segmentation

FreeSurfer 6.0 segmented the cortical and subcortical anatomy with a longitudinal pipeline [41-44]. We processed all the time points separately with the default FreeSurfer workflow and created an unbiased template from all the time points for each subject. Once this template was created, parcellations and segmentation were carried out at each time point initialized with common information from the within-subject template [42]. We calculated the percent change of the right hippocampus relative to the pre-treatment hippocampal volume.

### I. E-Field Modeling

The objective of E-field modeling in this study was to characterize the individual strength of the E-field delivered to the brain for a given electrode current amplitude. E-field modeling represents a single ECT pulse and does not include differences related to the temporal aspects of stimulation parameters (pulse width, train duration, and frequency), which were fixed for RUL ECT. The SimNIBS software (ver. 3.2.3 with headreco segmentation algorithm) was used to create a subject-specific, anatomically realistic volume conductor model [45]. The quasi-static approximation was assumed, which considers bioelectric currents in living tissues as stationary and resistive [46,47]. The T1- and T2-weighted scans were segmented into biological tissues and converted to a tetrahedral head mesh using Gmsh, a three-dimensional finite element (FE) mesh generator. Unique conductivity values for each tissue type were based on previous research: cerebrospinal fluid: (1.654 siemens/meter (S/m)), vitreous bodies (0.50 S/m), scalp (0.465 S/m), gray matter (0.275 S/m), white matter (0.126 S/m), spongy bone (0.025 S/m), and compact bone (0.0008 S/m) [45]. ECT electrodes were added to the head mesh in a RUL configuration (C2 and FT8 based on the 10-20 system). SimNIBS then used a FE solver to calculate the electric potentials and electric fields that correspond to the stimulation throughout the head mesh. We calculated E_{brain} as the 90th percentile of E-field magnitude in the whole brain for RUL electrode placement to avoid the influence of tissue boundary effects that could bias the absolute maximum E-field values [16]. E_{brain}/I is the E-field magnitude per unit stimulus current amplitude (in units of volts/meter (V/m) per milliampere (mA)). The E_{brain}/I ratio can then be multiplied by the electrode current during amplitude-determined seizure or during the latter 800 mA treatments to determine the electric field strength with the applied amplitude. We focused on E_{brain}/I as the locations of seizure generation, antidepressant response, and cognitive impairment are not known.

### J. Statistical Analyses

We restricted the analysis to treatments completed with RUL electrode placement. We performed summary statistics on all clinical and demographic measures and assessed longitudinal change with paired t-tests. We used linear regressions to assess the relationships of 1) E_{brain}/I on STₐ; 2) STₐ on right hippocampal volume change; and 3) E_{brain}/I on right hippocampal volume change. Covariates included age, sex, and treatment number (for right hippocampal volume change [48]) and the inclusion of interactions with Akaike Information Criterion. Regression diagnostics were consistent with model assumptions. We also assessed the relationships between STₐ, E_{brain}/I, and right hippocampal percent volume change with depression severity (percent change of IDS-C) and cognitive outcomes (change in DKEFS Letter and Category Fluency Summary Scores). Sex and TOPF standard score were included as covariates for cognitive outcomes (age was included in demographic-adjusted Scaled Scores). We present the results with right hippocampal volume change and clinical outcomes with E_{brain}/I with the qualification that E_{brain_800} is proportional to E_{brain}/I. We performed logistic regressions for significant antidepressant (electrode placement switch, responder/non-responder) and cognitive relationships (impaired/not impaired with dichotomization with ≤˜3 scaled score for letter, category fluency outcomes [28]). If logistic regression demonstrated a relationship, we performed receiver operating characteristic curves to determine the sensitivity and specificity at the empirical cut-point. Finally, we compared the regional E-field (i.e., Eᵣ₋ₕᵢₚₚₒ/I, E_{r-amygdala}/I) strength for 166 cortical and subcortical FreeSurfer regions with antidepressant and cognitive outcomes.

### K. General ECT Procedures:

ECT procedures follow the method described above in Example 1 (G), which is incorporated into this section in its entirety.

### Example 4: Clinical Trial to Apply Amplitude-Determined Seizure Titrations to Reduce the E-Field Variability

Using the methods described in Example 3 above, the relationships between amplitude-determined seizure-titration, E-field magnitude, and clinical outcomes were assessed in older depressed subjects (age range 50 to 80 years). Subjects received imaging, clinical, and neuropsychological assessment pre-, mid-, and post-ECT. STₐ was completed during the first treatment with a Soterix Medical 4X1 High Definition ECT Multi-channel Stimulation Interface (Investigation Device Exemption: G200123). Subsequent treatments were completed with right unilateral electrode placement (RUL) and 800 mA. E_{brain} for each subject was calculated as the 90^{th} percentile of E-field magnitude in the whole brain for right unilateral electrode placement. Twenty-nine subjects were included in the final analyses. E_{brain} was associated with STₐ. STₐ was associated with antidepressant outcomes at the mid-ECT assessment and bitemporal electrode placement switch. E_{brain} was associated with changes in category fluency with a large effect size. The relationship between STₐ and E_{brain} extends work from non-human primates and provides a validation step for ECT E-field modeling. Individualized amplitude based on E-field modeling or STₐ has the potential to eliminate the trial-and-error methods of ECT parameter selection and improve clinical outcomes.

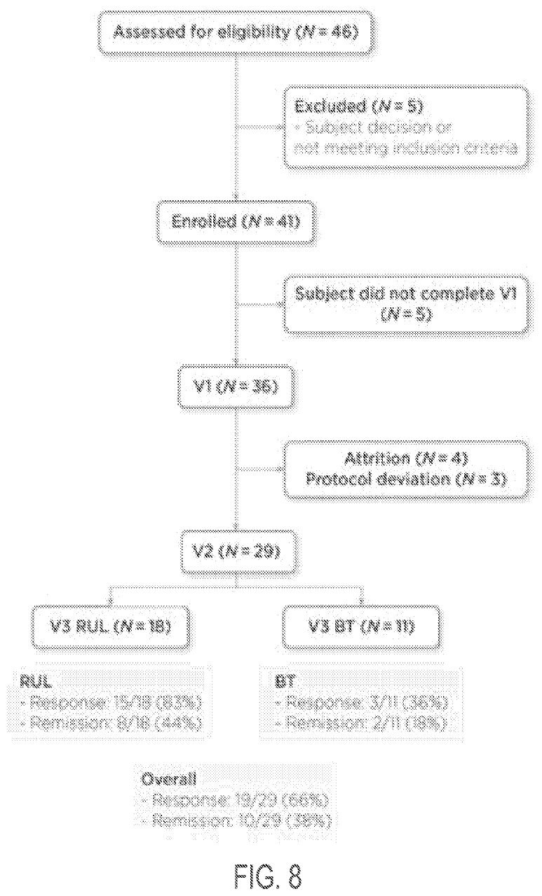

Forty-one subjects enrolled in study protocol from March 2021 to September 2022 consistent with pre-defined enrollment goals. Thirty-two subjects completed baseline (V1) assessment and received amplitude-determined seizure threshold titration. Three subjects were excluded from final analysis based on the following protocol deviations: pulse width error, amplitude titration error, and non-protocol determined switch to BT electrode placement. Five subjects received propofol when methohexital was unavailable, and they were included in the final analyses of twenty-nine subjects (FIG. 8). The demographic, clinical characteristics, antidepressant, and cognitive outcomes are summarized in Table 1. The average age and sex distribution of the study sample was 64.2 (+/−7.9 Standard deviation (SD)) years and 12 male/17 female.

TABLE I

| | Pre-ECT | | Post-ECT | | t-Statistic | |
|---|---|---|---|---|---|---|
| Clinical and demographic features | mean (SD) | | mean (SD) | | (p-value) | |
| Age +/− SD (years) | 64.2 | (7.9) | | | | |
| Sex: Male/Female | 12/17 | | | | | |
| Race: White/Black/Asian | 27/1/1 | | | | | |
| Ethnicity: Non-Hispanic/Hispanic | 24/5 | | | | | |
| Single episode/recurrent | 1/28 | | | | | |
| Psychotic/non-psychotic | 5/24 | | | | | |
| Episode duration (months) | 44.4 | (59.0) | | | | |
| Number of episodes | 4.3 | (3.0) | | | | |
| Age of onset (years) | 28.8 | (17.1) | | | | |
| Lifetime duration (years) | 17.2 | (16.2) | | | | |
| ECT Appropriateness Scale | 8.5 | (1.4) | | | | |
| Maudsley Treatment Failure | 2.4 | (1.3) | | | | |
| Education* | 5.8 | (1.7) | | | | |
| Antidepressants: SSRI/SNRI/TCA/NDRI/NaSSA/ | 7/12/3/2/2/3 | | | | | |
| SMS** | | | | | | |
| Antipsychotics/Mood Stabilizers/Benzodiazepines | 14/0/10 | | | | | |
| Amplitude determined seizure, Ebrain, seizure | | | | | | |
| duration | | | | | | |
| Amplitude-determined seizure (milliampere) | 312.3 | (112.9) | | | | |
| Ebrain (Volts/meter per milliampere) | 0.15 | (0.02) | | | | |
| EEG seizure duration titration (seconds) | 42.5 | (38.8) | | | | |
| EEG seizure duration 800 mA (seconds) | 38.9 | (18.8) | | | | |
| RUL treatment number | 8.1 | (2.8) | | | | |
| Depression Assessment | | | | | | |
| Inventory of Depressive Symptoms - Clinician Rated | 48.5 | (10.0) | 27.3 | (16.1) | 6.6 | (<0.001) |
| Cognitive Assessments | | | | | | |
| Montreal Cognitive Assessment | 23.9 | (3.8) | | | | |
| Test of Premorbid Functioning Standard Score Total | 108.6 | (13.9) | | | | |
| Delis Kaplan Executive Function System | | | | | | |
| Letter Fluency Scaled Score | 8.8 | (3.8) | 7.9 | (4.0) | 2.4 | (0.03) |
| Category Fluency Scaled Score | 9.2 | (4.6) | 7.7 | (3.9) | 2.7 | (0.01) |
| Category Switching Total Switching Accuracy | 8.6 | (4.1) | 8.3 | (3.8) | 0.4 | (0.66) |
| Scaled Score | | | | | | |
| Color-Word Interference Condition 2 Scaled Score | 9.3 | (3.1) | 8.3 | (3.8) | 2.2 | (0.04) |
| Color-Word Interference Condition 3 Scaled Score | 9.1 | (3.1) | 7.9 | (4.3) | 1.4 | (0.18) |
| Tower Total Achievement Scaled Score | 9.65 | (3.0) | 10.1 | (3.0) | −0.8 | (0.46) |
| California Verbal Learning Test -3^{rd} Edition | | | | | | |
| Trial 1 Free recall Correct Standard score | 4.4 | (1.4) | 10.7 | (3.4) | −11.3 | (<0.001) |
| Long delay free recall correct Standard score | 7.9 | (3.4) | 8.8 | (3.6) | −1.7 | (0.11) |
| Standard Score Summary - Trials I - 4 Correct | 32.3 | (12.3) | 37.9 | (10.9) | −3.1 | (0.005) |
| Sum of Scaled Scores | | | | | | |
| Delayed Recall Correct Sum of Scaled Scores | 23.6 | (9.3) | 25.3 | (10.1) | −1.2 | (0.23) |
| Total Recall Correct Sum of Scaled Scores | 55.9 | (20.0) | 63.2 | (19.6) | −2.5 | (0.02) |
| Dot Counting Test | | | | | | |
| Mean Ungrouped Time (seconds) | 7.3 | (2.0) | 8.7 | (3.1) | −2.6 | (0.01) |
| Mean Grouped Time (seconds) | 3.2 | (2.6) | 2.6 | (1.2) | 2.3 | (0.03) |
| E-Score | 11.5 | (3.6) | 12.6 | (4.0) | −1.4 | (0.17) |
| Wechsler Adult Intelligence Scale IV Digit Span | 10.4 | (3.3) | 10.0 | (1.9) | 0.8 | (0.39) |
| Scaled Score | | | | | | |

*Education, 1 = grade 6 or less, 2 = grade 7-12, 3 = graduated high school, 4 = part college or university, 5 = graduated 2-year college, 6 = graduated 4-year college, 7 = part graduate or professional school, 8 = completed graduate or professional school
**SSRI: select serotonin reuptake inhibitor, SNRI: serotonin-norepinephrine reuptake inhibitor, TCA: tricyclic antidepressant, NDRI: norepinephrine-dopamine reuptake inhibitor, NaSSA: noradrenergic and specific serotonergic antidepressant, SMS: serotonin modulator and stimulator

A. Amplitude-Determined Seizure (STₐ) and E_{brain}, and Right Hippocampal Volume Change.

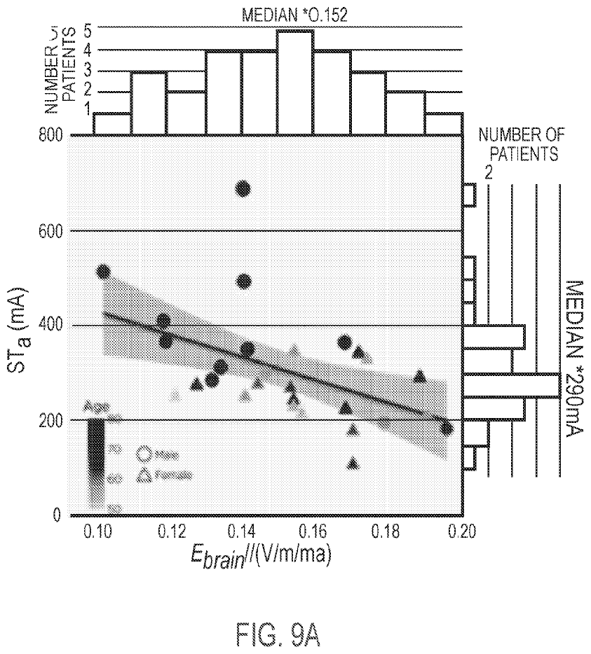

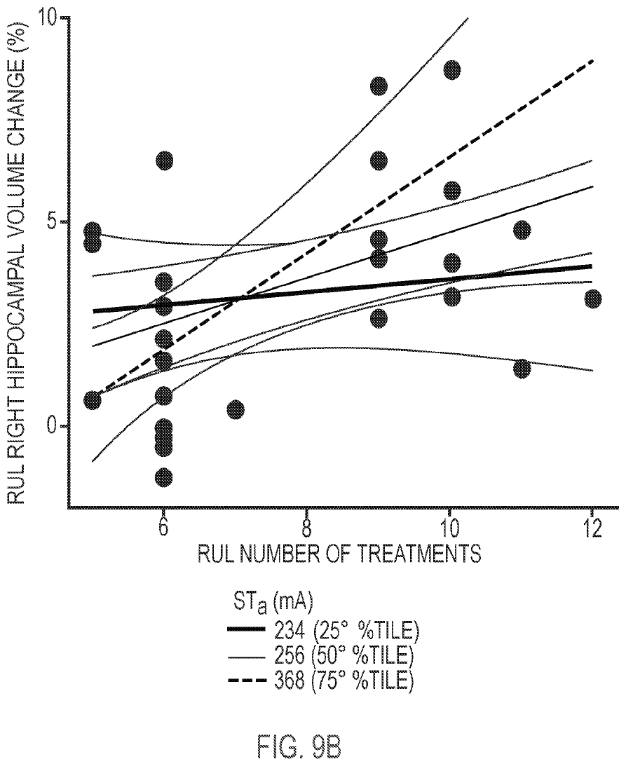

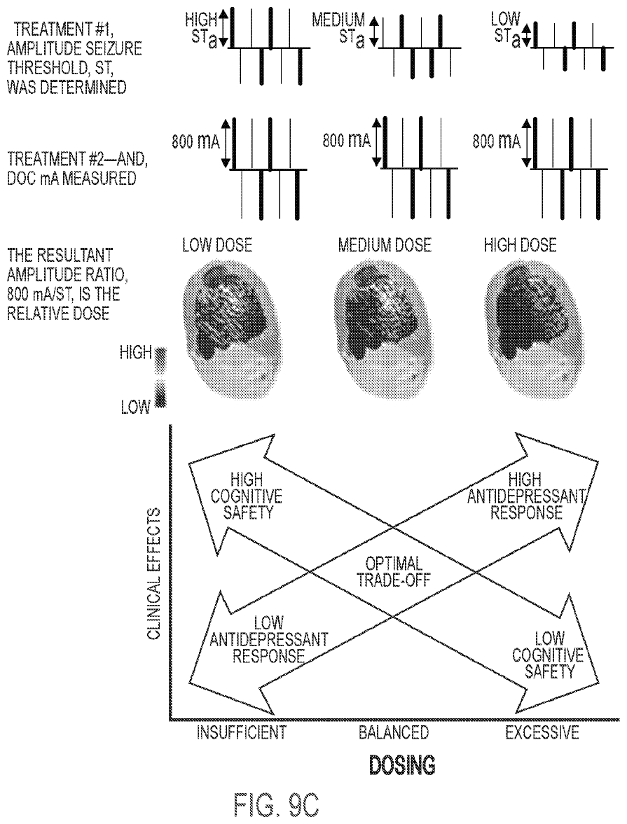

STₐ (312.34 mA, +/−113.07 SD, range: 120-686) and E_{brain}/I(0.15 V/m/mA, +/−0.02, range: 0.10-0.19) had considerable range (FIG. 9A). The average E_{brain} for amplitude-determined seizure (E_{brain_ST}) was 45.6 (+/−14.31) V/m. E_{brain}/I was associated with STₐ (β=−2074.16, t₂₅=−2.95, p=0.007, eta-squared=0.26). Age (β=5.61, t₂₅=2.55, p=0.02, eta-squared=0.21) but not sex (β=−45.47, t₂₅=−1.26, p=0.22, eta-squared=0.06) explained additional variance associated with STₐ. STₐ and treatment number interaction was associated with right hippocampal volume change (β=0.000075, t₂₁=2.24, p=0.04, eta-squared=0.24) such that lower STₐ had an earlier right hippocampal volume increase that plateaued with higher treatment number (FIG. 9B). E_{brain}/I and treatment number interaction was not associated with right hippocampal volume change (β=−0.21, t₂₁=−1.88, p=0.07, eta-squared=0.17).

### B. Antidepressant Outcomes

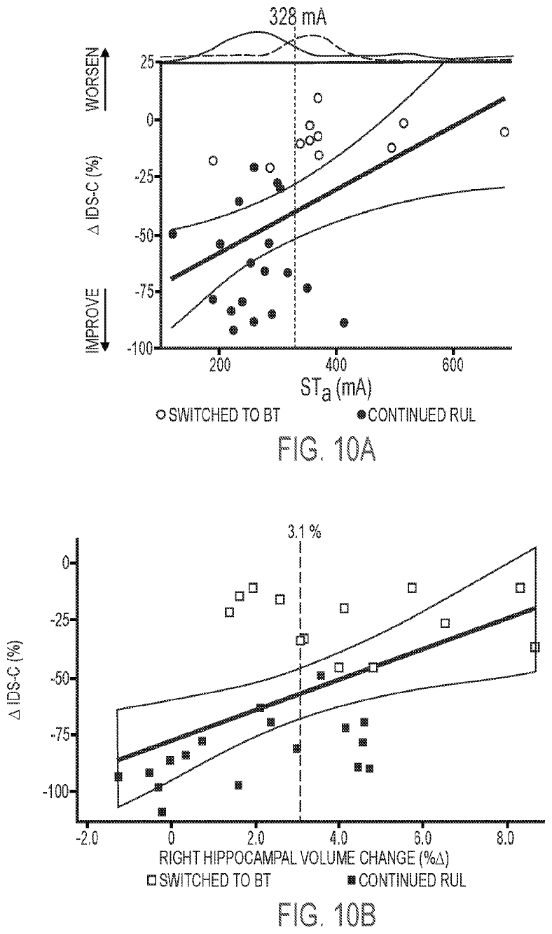

Increased STₐ was associated with poor antidepressant outcomes at V2 (β=−0.0012, t₂₅=−2.48, p=0.02, eta-squared=0.20) but not at the V3 assessment (β=−0.0012, t₂₅=−2.04, p=0.05, eta-squared=0.14) (FIG. 10A). STₐ did not differentiate RUL response/non-response outcomes (β=−0.013, z₂₅=−1.93, p=0.05) but did differentiate BT electrode placement switch (β=0.021, z₂₅=2.33, p=0.02, empirical cut point=328 mA with sensitivity=0.82 and specificity=0.89, area under the curve (AUC)=0.85). E_{brain}/I was not associated with antidepressant outcomes (β=0.016, t₂₅=0.01, p=0.99, eta-squared<0.01). Increased right hippocampal volume change was associated with improved antidepressant outcomes (β=5.27, t₂₃=2.37, p=0.03, eta-squared=0.20). Right hippocampal volume change differentiated RUL response/non-response outcomes (β=45.64, z₂₃=1.98, p=0.048, empirical cut point=3.1% with sensitivity=0.79 and specificity=0.67, AUC=0.73) and BT switch at V2 (β=−61.96, z₂₃=−2.06, p=0.04, empirical cut point=4.3% with sensitivity=0.18 and specificity=0.76, AUC=0.20) (FIG. 10B). Whole brain analysis comparing regional electric field strength and antidepressant outcomes did not reveal any relationships (uncorrected p>0.05).

### C. Cognitive Outcomes

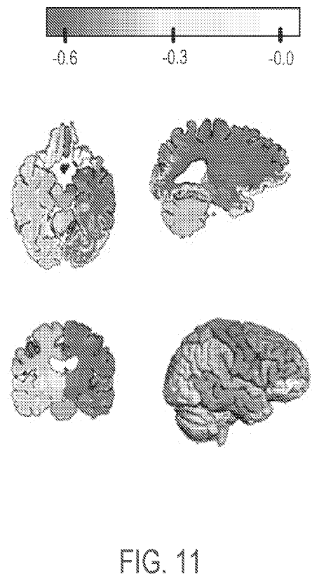

STₐ was not associated with changes in letter (β=0.0025, t₂₄=0.60, p=0.55, eta-squared=0.01) or category fluency (β=0.0090, t₂₄=1.61, p=0.12, eta-squared=0.10). E_{brain}/I was not associated with changes in letter fluency (β=−28.28, t₂₄=−1.62, p=0.12, eta-squared=0.10) but was associated with changes in category fluency with a large effect size (β=−68.74, t₂₄=−3.15, p=0.004, eta-squared=0.29). E_{brain}/I did not differentiate dichotomous category fluency outcomes (β=33.75, z₂₄=1.72, p=0.09). Right hippocampal volume change was not associated with letter (β=−33.46, t₂₃=−1.85, p=0.08, eta-squared=0.13) or category fluency change (β=−47.26, t₂₃=−1.84, p=0.08, eta-squared=0.13). In an exploratory analysis, 16/29 subjects had impaired longitudinal performance with either letter or category fluency. E_{brain}/I differentiated dichotomous combined letter and category fluency outcomes (β=57.15, z₂₃=2.15, p=0.03, empirical cut point=0.15 V/m/mA or 119 V/m at 800 mA with sensitivity=0.69 and specificity=0.69, AUC=0.69). Whole brain analysis comparing regional electric field strength and category fluency outcomes revealed widespread right hemisphere associations (FIG. 11). From the 54 cortical parcellations that demonstrated a relationship with category fluency (p_{FDR}<0.05), 44 parcellations were from the right hemisphere. Right subcortical segmentations included the right pallidum, amygdala, nucleus accumbens, diencephalon, putamen, thalamus and hippocampus.

### D. Discussion

This investigation used a unique design with amplitude-determined seizure titration at the first treatment followed by fixed 800 mA for subsequent treatments. Pulse number (20 Hz frequency and 8 s pulse train duration) was held constant at 160 pulse pairs for the STₐ titration and the 800 mA treatments. Both STₐ and E_{brain}/I had a wide range, which challenges the long-standing use of fixed amplitude ECT. STₐ increased with age, which is consistent with the observations made by Liberson when “brief stimulus therapy” was first experimented in the 1940s [49]. The relationship between STₐ and E_{brain}/I extends work from preclinical models [15,16] and provides a validation step for ECT E-field modeling. Despite their relationship, STₐ and E_{brain}/I had different relationships with antidepressant and cognitive outcomes. The antidepressant and cognitive outcomes in relation to STₐ may be understood as a ratio between STₐ and subsequent 800 mA treatments (800 mA/STₐ) (FIG. 9C). Lower ratios (e.g., 800 mA/686 mA) may be inadequate to achieve the necessary “suprathreshold” dosing for antidepressant response. Higher STₐ (and hence lower 800 mA/STₐ ratio) was associated with the BT switch at V2 as determined by <25% change from baseline depression severity. Higher E_{brain}/I was associated with worse cognitive outcomes as measured by longitudinal change in category fluency. E_{brain}/I at the mid-point of the distribution (0.15 V/m/mA) differentiated cognitive impairment with 800 mA amplitude. STₐ and treatment number interaction was related to right hippocampal volume change, and right hippocampal volume change was associated with antidepressant outcomes (bitemporal switch and RUL response). In the following sections, we provide context for these findings, strengths and limitations of this approach, and potential implications for the practice of ECT dosing.

### Antidepressant Outcomes

Our antidepressant results demonstrated improved efficacy with “suprathreshold” treatments. In current clinical practice, suprathreshold treatments are defined in the context of pulse number (i.e., pulse train duration and frequency). The minimum number of pulses for a fixed amplitude and pulse width determines the seizure threshold. The suprathreshold multiplier (typically six-times seizure threshold for RUL ECT) determines the individualized pulse number necessary for antidepressant efficacy [50]. In contrast, the suprathreshold specifier can also be applied to the stimulus current amplitude. Our findings indicate that for ECT with 800 mA fixed amplitude to be effective, the inflection point for STₐ is approximately 330 mA, corresponding to fixed amplitude/STₐ ratio of 800 mA/330 mA or ˜2.5. Higher STₐ (>330 mA) is associated with inadequate antidepressant response with subsequent treatments completed at 800 mA resulting in a protocol-determined switch to BT electrode placement. When the STₐ is greater than 330 mA, increased amplitude (>800 mA) may improve antidepressant efficacy. Alternatively, an electrode placement switch from RUL to bitemporal may also provide the necessary suprathreshold dose for antidepressant efficacy in the context of high STₐ.

Previous research with E-field strength and antidepressant outcomes has been mixed [14,17,51]. In this current study sample, E_{brain}/I was unrelated to antidepressant outcomes. We also did not replicate the previously identified relationship between right hippocampal E-field strength (Eₕᵢₚₚₒ/j) and right hippocampal volume change [14]. Differences between these two investigations include the focus on RUL (previous investigation included RUL and BT) and 800 mA (previous investigation included 600 and 700 mA). Despite not demonstrating the E_{brain}/I and hippocampal volume relationship, right hippocampal volume change was associated with antidepressant outcome including response criteria and bitemporal electrode placement switch. Larger investigations are necessary to disentangle the effect of E_{brain}/I from ECT treatment number and other parameters (i.e., pulse width, stimulation time) on hippocampal volume change and to explore potential moderating effects of structural and functional changes between E_{brain}/I and antidepressant outcomes. In contrast, STₐ may capture additional information such as cortical excitability not included in E_{brain}/I that strengthens the relationships to antidepressant outcomes [52] or age-related changes in conductivity (i.e., white matter disease) not presently included in E-field modeling approaches [53].

### Cognitive Outcomes

The DKEFS Category and Letter Fluency tests were sensitive to RUL-mediated changes in cognitive performance. Higher E_{brain}/I was associated with worse category fluency performance. In contrast, STₐ was unrelated with cognitive outcomes. The strong association between E_{brain}/I and cognitive outcomes replicates our previous work and adds support for the role of E_{brain}/I in ECT dosing [14,54]. In this sample, an E_{brain}/I of 0.15 V/m/mA was the maximal associated with stable cognitive performance with traditional fixed amplitude 800 mA dosing. When E_{brain}/I is greater than 0.15 V/m/mA (120 V/m at 800 mA), decreased amplitude (<800 mA) may reduce cognitive risk. The widespread right hemisphere associations between E_{brain}/I and cognitive outcomes did not identify a specific anatomic “anti-target” amenable to changes in electric field geometry to improve the focality of treatment to prevent cognitive impairment. In contrast, an individualized stimulus amplitude determined prior to treatment initiation has the potential to improve cognitive outcomes.

### ECT Dosing

Currently, most ECT clinicians implement a trial-and-error approach to parameter selection, initially favoring reduced cognitive risk (RUL and ultrabrief pulse width) before advancing to other parameters with increased cognitive risk (bitemporal and brief pulse width) [19]. This trial-and-error approach may still expose patients to increased cognitive risk with RUL (i.e., patient has high E_{brain}/I) and may miss the optimal dose for antidepressant response without cognitive risk. The fixed current amplitude approach fails to consider individual anatomic variability resulting in variable brain E-field and sub-optimal clinical outcomes. An individualized amplitude that is sufficient for antidepressant effect with greater cognitive safety can be determined prior to treatment initiation. Individualized current amplitude can also eliminate the trial-and-error methods of ECT parameter selection thus reducing the overall number of treatments of the ECT series and expediting antidepressant response.

The first approach to individualized amplitude uses STₐ during the first treatment with a suprathreshold multiplier for subsequent treatments. Based on our data, the highest STₐ associated with antidepressant response at 800 mA was 330 mA, indicating 2.5×STₐ as an appropriate suprathreshold current multiplier. Similar to pulse number titrations, the first treatment would be sub-therapeutic with no expectations of antidepressant efficacy. The second approach uses pre-treatment E-field modeling. The optimal individualized current amplitude can be determined by dividing the optimal E-field strength by the individual E_{brain}/I. The optimal E-field strength is sufficient to induce an antidepressant effect without cognitive impairment. The relationship between E_{brain} and cognitive outcome provides an upper threshold for optimal dosing (110-120 V/m). E-field informed ECT would eliminate the dose finding approach to the first treatment and start with therapeutic stimulation but would require MRI acquisition and processing. Yet another potential approach is to carry out a non-convulsive motor threshold titration through the ECT electrodes and leverage its correlation with STₐ and E_{brain}/I observed in preclinical studies [15,59]. After amplitude individualization, further research should seek to identify optimal frequency and number of pulses and determine if they vary across subjects. Any of these approaches to ECT current individualization would require commercial device development for fine amplitude adjustments (˜1 mA) and lower starting amplitude (100 mA), although such devices have been available for experimental studies [60]. Regardless of approach, individualized amplitude has the potential to advance neuroscience-based ECT dosing strategies and optimize both antidepressant and cognitive outcomes.

The following statements are intended to describe and summarize various embodiments of the invention according to the foregoing description in the specification.

### Statements:

1. A method of electroconvulsive therapy (ECT) for a patient's brain to treat a neuropsychiatric disorder, comprising:
determining an individualized amplitude for the patient by performing an amplitude-determined seizure-titration procedure on the patient's brain,
wherein the amplitude-determined seizure-titration procedure comprises stimulating the patient's brain with increasing amplitudes until seizure activity is initiated; and
wherein the amplitude at which seizure activity is initiated is the seizure-determined amplitude or STₐ for the patient;
   multiplying the patient's STₐ by a multiplier value to determine the patient's
individualized amplitude; and
applying the individualized amplitude to the patient's brain with a fixed placement of extracranial electrodes to improve the neuropsychiatric disorder with reduced cognitive impairment.
   2. The method of statement 1, wherein the amplitude-determined seizure-titration procedure occurs at a first treatment session with the patient.
   3. The method of statement 2, wherein applying the individualized amplitude to the patient's brain occurs at one or more subsequent treatments.
   4. The method of statements 1 and 3, wherein the multiplier value is approximately 2 to approximately 3.
   5. The method of statements 1 and 3, wherein the multiplier value is approximately 2.5.
   6. The method of statements 1 and 3, wherein the individualized amplitude is applied to the patient's brain with right unilateral electrode placement, a pulse width of approximately 0.3 to approximately 1.0 ms, a frequency of approximately 20 to approximately 120 hertz, and a pulse train duration of approximately 8 seconds).
   7. The method of statement 1, wherein stimulating the patient's brain comprises placement of extracranial electrodes on the patient's head and applying an amplitude starting at approximately 100 mA with incremental increases in amplitude until seizure activity occurs.
   8. The method of statement 7, wherein the increase in amplitude is in increments of 1 mA, 25 mA, 50 mA, or 75 mA.
   9. The method of statement 1, wherein the initiation of seizure activity is determined by right lower extremity motor activity, EEG activity for 20 seconds, or both.
   10. The method of statement 3, wherein the number of subsequent treatments is based on improving a neuropsychiatric disorder without cognitive impairment of the patient.
   11. The method of statement 10, wherein the neuropsychiatric disorder is major depressive disorder (MDD).
   12. The method of statement 10, wherein the neuropsychiatric disorder is any one or more of schizophrenia, schizoaffective disorder, or bipolar disorder.
   13. The method of statement 10, wherein the neuropsychiatric disorder meets a clinical indication for treatment with ECT.
   14. The method of statement 1, wherein the patient is aged from 18 years or older.
   15. The method of statement 1, further comprising placing the patient under general anesthesia prior to applying the optimal individualized current amplitude to the patient's brain.
   16. The method of statement 10, wherein the cognitive impairment comprises one or more of amnesia, executive dysfunction, or verbal dysfluency.
   17. The method of statement 1, wherein the improvement to the neuropsychiatric disorder comprises an antidepressant effect as measured by an improvement in the patient's score on a depression rating scale.
   18. The method of statement 1, wherein the patient receives the ECT approximately three times per week for approximately four weeks.
   19. A method of electroconvulsive therapy (ECT) to treat a neuropsychiatric disorder in a patient, comprising:
determining an individualized amplitude for the patient by performing an amplitude-determined seizure-titration procedure on the patient's brain with right unilateral (RUL) electrode placement at a first treatment,
wherein the amplitude-determined seizure-titration procedure comprises stimulating the patient's brain with increasing amplitudes with a fixed frequency and pulse train duration until seizure activity is initiated; and
wherein the amplitude at which seizure activity is initiated is the seizure-determined amplitude or STₐ for the patient;
   applying the STₐ multiplied by a multiplier value to the patient's brain at one or
more subsequent treatments to improve the neuropsychiatric disorder with reduced cognitive impairment, wherein the multiplier value is approximately 2 to approximately 3.
   20. The method of statement 19, wherein the initiation of seizure activity is determined by right lower extremity motor activity, EEG activity for 20 seconds, or both.
   21. The method of statement 19, wherein the increase in amplitude is in increments of 1 mA, 25 mA, 50 mA, or 75 mA.
   22. The method of statement 19, wherein frequency is approximately 20 to approximately 120 hertz and the pulse train duration is approximately 8 seconds.
   23. The method of statement 19, wherein a range of amplitudes at which seizure activity occurs is from approximately 100 mA to approximately 700 mA.
   24. A method of electroconvulsive therapy (ECT) for a patient's brain, comprising:
determining an optimal individualized current amplitude for the patient by dividing an optimal E-field strength (optimal E_{brain}) by a baseline E-field strength (baseline E_{brain}) of the patient's brain,
wherein the optimal E_{brain} is an E-field strength sufficient to induce
an antidepressant effect without cognitive impairment; and
wherein the baseline E_{brain} is determined by finite element
modeling using structural MRI of the patient's brain with a fixed placement of extracranial electrodes and comprises at least a 90th percentile of E-field magnitudes from all voxels in the patient's brain, and
   applying the optimal individualized current amplitude to the patient's brain
with the fixed placement of the extracranial electrodes,
wherein the optimal individualized current amplitude induces a seizure activity that has an antidepressant effect and reduces an adverse cognitive effect on the patient's brain.
   25. The method of statement 23, wherein the optimal E_{brain} is approximately 110 Volts/meter to approximately 120 Volts/meter.
   26. The method of statement 23, wherein E_{brain} is based on an E-field of the patient's whole brain.
   27. The method of statement 23, wherein the finite element modeling comprises segmentation of the brain, extracranial electrode placement on the patient's scalp, tessellation of volume into a mesh, and determining the patient's baseline E_{brain}.
   28. The method of statement 27, wherein the electrode placement on the patient's scalp determines an E-field geometric shape.
   29. The method of statement 28, wherein a current amplitude determines the E-field magnitude within the E-field shape.
   30. The method of statement 23, wherein the 90th percentile of E-field magnitudes ranges from approximately 0.1 to approximately 0.2 Volts/meter per milliampere of current.
   31. The method of statement 23, wherein the ECT treats a neuropsychiatric disorder in the patient.
   32. The method of statement 31, wherein the neuropsychiatric disorder is major depressive disorder (MDD).
   33. The method of statement 31, wherein the neuropsychiatric disorder is any one or more of schizophrenia, schizoaffective disorder, or bipolar disorder.
   34. The method of statement 31, wherein the neuropsychiatric disorder meets a clinical indication for treatment with ECT.
   35. The method of statement 23, wherein the patient is aged from 18 years or older.
   36. The method of statement 23, further comprising placing the patient under general anesthesia prior to applying the optimal individualized current amplitude to the patient's brain.
   37. The method of statement 23, wherein the adverse cognitive effect comprises one or more of amnesia, executive dysfunction, or verbal dysfluency.
   38. The method of statement 23, wherein the antidepressant effect comprises an improvement in the patient's score on a depression rating scale.
   39. The method of statement 23, wherein the patient receives the ECT approximately three times per week for approximately four weeks.
   40. The method of statement 23, wherein the optimal individualized current amplitude is applied to the patient's brain at a pulse width of approximately 0.3 to approximately 1.0 milliseconds milliseconds.
   41. The method of statement 40, wherein the optimal individualized current amplitude is applied to the patient's brain for a pulse train duration of approximately 0.5 to approximately 8 seconds.
   42. The method of statement 23, wherein the optimal individualized current amplitude ranges from approximately 600 mA to approximately 1200 mA.
   43. A method of determining an optimal individualized current amplitude for a patient for use in electroconvulsive therapy (ECT) to the patient's brain, comprising:
determining a baseline E-field strength (baseline E_{brain}) of the patient's brain by
   finite element modeling using structural MRI of the patient's brain with a fixed placement of extracranial electrodes,
   wherein the baseline E_{brain} comprises at least a 90th percentile of E-field magnitudes from all voxels in the patient's brain, and
dividing an optimal E-field strength (optimal E_{brain}) by the patient's baseline E_{brain}
   to produce the optimal individualized current amplitude for the patient,
   wherein the optimal E_{brain} is an E-field strength sufficient to induce
   an antidepressant effect without cognitive impairment.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The technology illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a plant” or “a seed” or “a cell” includes a plurality of such plants, seeds or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The technology has been described broadly and generically herein. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein. In addition, where features or aspects of the technology are described in terms of Markush groups, those skilled in the art will recognize that the technology is also thereby described in terms of any individual member or subgroup of members of the Markush group. The Abstract is provided to allow the reader to quickly ascertain the nature and gist of the technical disclosure. The Abstract is submitted with the understanding that it will not be used to interpret or limit the scope or meaning of the claims.

### REFERENCES

1 U K ECT Review Group. Efficacy and safety of electroconvulsive therapy in depressive disorder: a systematic review and meta-analysis. Lancet. 2003; 361:799-808.
2 McClintock S M, Choi J, Deng Z D, Appelbaum L G, Krystal A D, Lisanby S H. Multifactorial determinants of the neurocognitive effects of electroconvulsive therapy. J ECT. 2014; 30(2):165-76.
3 Semkovska M, McLoughlin D M. Objective cognitive performance associated with electroconvulsive therapy for depression: a systematic review and meta-analysis. Biological psychiatry. 2010; 68(6):568-77.
4 Semkovska M, Keane D, Babalola O, McLoughlin D M. Unilateral brief-pulse electroconvulsive therapy and cognition: effects of electrode placement, stimulus dosage and time. Journal of psychiatric research. 2011; 45(6):770-80.
5 Obbels J, Verwijk E, Vansteelandt K, Dols A, Bouckaert F, Schouws S, et al. Long-term neurocognitive functioning after electroconvulsive therapy in patients with late-life depression. Acta psychiatrica Scandinavica. 2018; 138(3):223-31.
6 Breggin P R. Toxic psychiatry: why therapy, empathy, and love must replace the drugs, electroshock, and biochemical theories of the “new psychiatry”. 1st ed. St. Martin's Press: New York; 1991.
7 Breggin P R. Brain Disabling Treatments in Psychiatry: Drugs, Electroshock, and the Psychopharmaceutical Complex. Springer Publishing Company: New York; 2007.
8 Sterling P. ECT damage is easy to find if you look for it. Nature. 2000; 403(6767):242.
9 Frank L R. Electroshock: a crime against the spirit. Ethical Hum Sci Serv. 2002; 4(1):63-71.
10 Vasavada M M, Leaver A M, Njau S, Joshi S H, Ercoli L, Hellemann G, et al. Short- and Long-term Cognitive Outcomes in Patients With Major Depression Treated With Electroconvulsive Therapy. J ECT. 2017; 33(4):278-85.
11 Peterchev A V, Rosa M A, Deng Z D, Prudic J, Lisanby S H. Electroconvulsive therapy stimulus parameters: rethinking dosage. J ECT. 2010; 26(3):159-74.
12 Deng Z D, Lisanby S H, Peterchev A V. Effect of anatomical variability on electric field characteristics of electroconvulsive therapy and magnetic seizure therapy: a parametric modeling study. IEEE Trans Neural Syst Rehabil Eng. 2015; 23(1):22-31.
13 Abbott C C, Quinn D, Miller J, Ye E, Iqbal S, Lloyd M, et al. Electroconvulsive Therapy Pulse Amplitude and Clinical Outcomes. Am J Geriatr Psychiatry. 2021; 29(2):166-78.
14 Deng Z D, Argyelan M, Miller J, Quinn D K, Lloyd M, Jones T R, et al. Electroconvulsive therapy, electric field, neuroplasticity, and clinical outcomes. Molecular psychiatry. 2022; 27(3):1676-82.
15 Peterchev A V, Krystal A D, Rosa M A, Lisanby S H. Individualized Low-Amplitude Seizure Therapy: Minimizing Current for Electroconvulsive Therapy and Magnetic Seizure Therapy. Neuropsychopharmacology. 2015; 40(9):2076-84.
16 Lee W H, Lisanby S H, Laine A F, Peterchev A V. Minimum Electric Field Exposure for Seizure Induction with Electroconvulsive Therapy and Magnetic Seizure Therapy. Neuropsychopharmacology. 2017; 42(6):1192-200.
17 Argyelan M, Oltedal L, Deng Z D, Wade B, Bikson M, Joanlanne A, et al. Electric field causes volumetric changes in the human brain. Elife. 2019; 8.
18 First M B, Spitzer R L, Gibbon M, Williams J B W. Structured Clinical Interview for DSM-IV-TR Axis I Disorders, Research Version, Patient Edition. New York State Psychiatric Institute, Biomedical Research: New York, New York; 2002.
19 American Psychiatric Association. The Practice of Electroconvulsive Therapy: Recommendations for Treatment, Training, and Privileging. Second ed. American Psychiatric Association: Washington, D.C.; 2001.
20 Pluijms E M, Kamperman A M, Hoogendijk W J, Birkenhager T K, van den Broek W W. Influence of an adjuvant antidepressant on the efficacy of electroconvulsive therapy: A systematic review and meta-analysis. Aust N Z J Psychiatry. 2020:4867420952543.
21 Rush A J, Gullion C M, Basco M R, Jarrett R B, Trivedi M H. The Inventory of Depressive Symptomatology (IDS): psychometric properties. Psychological medicine. 1996; 26(3):477-86.
22 Fekadu A, Wooderson S, Donaldson C, Markopoulou K, Masterson B, Poon L, et al. A multidimensional tool to quantify treatment resistance in depression: the Maudsley staging method. The Journal of clinical psychiatry. 2009; 70(2):177-84.
23 Kellner C H, Popeo D M, Pasculli R M, Briggs M C, Gamss S. Appropriateness for electroconvulsive therapy (ECT) can be assessed on a three-item scale. Medical hypotheses. 2012; 79(2):204-6.
24 Oldfield R C. The assessment and analysis of handedness: the Edinburgh inventory. Neuropsychologia. 1971; 9(1):97-113.
25 Wechsler D. Test of Premorbid Functioning. The Psychological Corporation: San Antonio, TX; 2009.
26 Nasreddine Z S, Phillips N A, Bedirian V, Charbonneau S, Whitehead V, Collin I, et al. The Montreal Cognitive Assessment, MoCA: A Brief Screening Tool For Mild Cognitive Impairment. J Am Geriatr Soc. 2005; 53(4):695-99.
27 Rossetti H C, Lacritz L H, Cullum C M, Weiner M E. Normative data for the Montreal Cognitive Assessment (MoCA) in a population-based sample. Neurology. 2011; 27(77):1272-75.
28 Delis D C, Kaplan E, J K. Delis Kaplan Executive Function System. The Psychological Corporation: San Antonio, TX; 2001.
29 Yochim B, Baldo J, Nelson A, Delis D C. D-KEFS Trail Making Test performance in patients with lateral prefrontal cortex lesions. J Int Neuropsychol Soc. 2007; 13:704-09.
30 Latzman R D, Markon K E. The factor structure and age-related factorial invariance of the Delis-Kaplan Executive Function System (D-KEFS). Assessment. 2010; 17:172-84.
31 Delis D C, Kramer J H, Kaplan E, Holdnack J. Reliability and validity of the Delis-Kaplan Executive Function System: An update. J Int Neuropsychol Soc. 2004; 10:301-03.
32 Mitchell M, Miller L S. Prediction of functional status in older adults: The ecological validity of four Delis-Kaplan Executive Function System tests. J Clin Exp Neuropsychol. 2008; 6:683-90.
33 Delis D C, Kramer J H, Kaplan E, Ober B A. California Verbal Learning Test-3 (Third Edition). The Psychological Corporation: San Antonio, TX; 2017.
34 Boone K B, Lu P, Herzberg D. Rey Dot Counting Test. Western Psychological Services: Los Angeles; 2002.
35 Wechsler D. Wechsler Adult Intelligence Scale—Fourth Edition. American Psychological Association: 750 First Street NE, Washington, DC 20002-4242; 2008.
36 d'Elia G. Unilateral electroconvulsive therapy. Acta Psychiatr Scand Suppl. 1970; 215:1-98.
37 Weaver L A, Jr., Ives J, Williams R. Studies in brief-pulse electroconvulsive therapy: the voltage threshold, interpulse interval, and pulse polarity parameters. Biological psychiatry. 1982; 17(10):1131-43.
38 Devanand D P, Lisanby S H, Nobler M S, Sackeim H A. The relative efficiency of altering pulse frequency or train duration when determining seizure threshold. J ECT. 1998; 14(4):227-35.
39 Spaans H P, Verwijk E, Comijs H C, Kok R M, Sienaert P, Bouckaert F, et al. Efficacy and cognitive side effects after brief pulse and ultrabrief pulse right unilateral electroconvulsive therapy for major depression: a randomized, double-blind, controlled study. The Journal of clinical psychiatry. 2013; 74(11):e1029-36.
40 Tor P C, Bautovich A, Wang M J, Martin D, Harvey S B, Loo C. A Systematic Review and Meta-Analysis of Brief Versus Ultrabrief Right Unilateral Electroconvulsive Therapy for Depression. The Journal of clinical psychiatry. 2015; 76(9):e1092-8.
41 Fischl B. FreeSurfer. NeuroImage. 2012; 62(2):774-81.
42 Reuter M, Schmansky N J, Rosas H D, Fischl B. Within-subject template estimation for unbiased longitudinal image analysis. NeuroImage. 2012; 61(4):1402-18.
43 Reuter M, Fischl B. Avoiding asymmetry-induced bias in longitudinal image processing. NeuroImage. 2011; 57(1):19-21.
44 Reuter M, Rosas H D, Fischl B. Highly accurate inverse consistent registration: a robust approach. NeuroImage. 2010; 53(4):1181-96.
45 Saturnino G B, Antunes A, Thielscher A. On the importance of electrode parameters for shaping electric field patterns generated by tDCS. NeuroImage. 2015; 120:25-35.
46 Plonsey R, Heppner D B. Considerations of quasi-stationarity in electrophysiological systems. Bull Math Biophys. 1967; 29(4):657-64.
47 Schwan H P, Kay C F. Capacitive properties of body tissues. Circ Res. 1957; 5(4):439-43.
48 Oltedal L, Narr K L, Abbott C, Anand A, Argyelan M, Bartsch H, et al. Volume of the Human Hippocampus and Clinical Response Following Electroconvulsive Therapy. Biological psychiatry. 2018; 84(8):574-81.
49 Liberson W T. Brief stimulus therapy; physiological and clinical observations. The American journal of psychiatry. 1948; 105(1):28-39.
50 Sackeim H A, Prudic J, Nobler M S, Fitzsimons L, Lisanby S H, Payne N, et al. Effects of pulse width and electrode placement on the efficacy and cognitive effects of electroconvulsive therapy. Brain Stimul. 2008; 1(2):71-83.
51 Fridgeirsson E A, Deng Z D, Denys D, van Waarde J A, van Wingen G A. Electric field strength induced by electroconvulsive therapy is associated with clinical outcome. Neuroimage Clin. 2021; 30:102581.
52 Casarotto S, Canali P, Rosanova M, Pigorini A, Fecchio M, Mariotti M, et al. Assessing the effects of electroconvulsive therapy on cortical excitability by means of transcranial magnetic stimulation and electroencephalography. Brain Topogr. 2013; 26(2):326-37.
53 Indahlastari A, Albizu A, Boutzoukas E M, O'Shea A, Woods A J. White matter hyperintensities affect transcranial electrical stimulation in the aging brain. Brain Stimul. 2021; 14(1):69-73.
54 Qi S, Calhoun V D, Zhang D, Miller J, Deng Z D, Narr K L, et al. Links between electroconvulsive therapy responsive and cognitive impairment multimodal brain networks in late-life major depressive disorder. BMC Med. 2022; 20(1):477.
55 Unal G, Poon C, FallahRad M, Thahsin M, Argyelan M, Bikson M. Quasi-static pipeline in electroconvulsive therapy computational modeling. Brain Stimul. 2023; 16(2):607-18.
56 Sartorius A. Electric field distribution models in ECT research. Molecular psychiatry. 2022; 27(9):3571-72.
57 Unal G, Swami J K, Canela C, Cohen S L, Khadka N, FallahRad M, et al. Adaptive current-flow models of ECT: Explaining individual static impedance, dynamic impedance, and brain current density. Brain Stimul. 2021; 14(5):1154-68.
58 Puonti O, Van Leemput K, Saturnino G B, Siebner H R, Madsen K H, Thielscher A. Accurate and robust whole-head segmentation from magnetic resonance images for individualized head modeling. NeuroImage. 2020; 219:117044.
59 Lee W H, Lisanby S H, Laine A F, Peterchev A V. Electric Field Model of Transcranial Electric Stimulation in Nonhuman Primates: Correspondence to Individual Motor Threshold. IEEE Trans Biomed Eng. 2015; 62(9):2095-105.
60 Nahas Z, Short B, Burns C, Archer M, Schmidt M, Prudic J, et al. A feasibility study of a new method for electrically producing seizures in man: focal electrically administered seizure therapy [FEAST]. Brain Stimul. 2013; 6(3):403-8.

1-43. (canceled)

44. A method of electroconvulsive therapy (ECT) for a patient's brain to treat a neuropsychiatric disorder, comprising:
determining an individualized amplitude for the patient by performing an amplitude-determined seizure-titration procedure on the patient's brain, wherein the amplitude-determined seizure-titration procedure comprises stimulating the patient's brain with increasing amplitudes until seizure activity is initiated, and wherein the amplitude at which seizure activity is initiated is the seizure-determined amplitude or STₐ for the patient;
multiplying the patient's STₐ by a multiplier value to determine the patient's individualized amplitude; and
applying the individualized amplitude to the patient's brain with a fixed placement of extracranial electrodes to improve the neuropsychiatric disorder.

45. The method of claim 44, wherein the amplitude-determined seizure-titration procedure occurs at a first treatment session with the patient, and wherein applying the individualized amplitude to the patient's brain occurs at one or more subsequent treatments.

46. The method of claim 45, wherein the number of subsequent treatments is based on improving a neuropsychiatric disorder without cognitive impairment of the patient, wherein the cognitive impairment comprises one or more of amnesia, executive dysfunction, or verbal dysfluency.

47. The method of claim 44, wherein the multiplier value is approximately 2 to approximately 3.

48. The method of claim 44, wherein the individualized amplitude is applied to the patient's brain with right unilateral electrode placement, a pulse width of approximately 0.3 to approximately 1.0 ms, a frequency of approximately 20 to approximately 120 hertz, and a pulse train duration of approximately 8 seconds.

49. The method of claim 44, wherein stimulating the patient's brain comprises placement of extracranial electrodes on the patient's head and applying an amplitude starting at approximately 100 mA with incremental increases in amplitude until seizure activity occurs.

50. The method of claim 44, wherein the initiation of seizure activity is determined by right lower extremity motor activity, EEG activity for 20 seconds, or both.

51. The method of claim 44, wherein the neuropsychiatric disorder is any one or more of major depressive disorder (MDD), schizophrenia, schizoaffective disorder, or bipolar disorder.

52. The method of claim 44, wherein the amplitude-determined seizure-titration procedure comprises stimulating the patient's brain with increasing amplitudes with a fixed frequency and pulse train duration until seizure activity is initiated.

53. The method of claim 52, wherein the frequency is approximately 20 to approximately 120 hertz and the pulse train duration is approximately 8 seconds, and wherein a range of amplitudes at which seizure activity occurs is from approximately 100 mA to approximately 700 mA.

54. A method of electroconvulsive therapy (ECT) for a patient's brain, comprising:
determining an optimal individualized current amplitude for the patient by dividing an optimal E-field strength (optimal E_{brain}) by a baseline E-field strength (baseline E_{brain}) of the patient's brain, wherein the optimal E_{brain} is an E-field strength sufficient to induce an antidepressant effect without cognitive impairment, and wherein the baseline E_{brain} is determined by finite element modeling using structural MRI of the patient's brain with a fixed placement of extracranial electrodes and comprises at least a 90th percentile of E-field magnitudes from all voxels in the patient's brain; and
applying the optimal individualized current amplitude to the patient's brain with the fixed placement of the extracranial electrodes, wherein the optimal individualized current amplitude induces a seizure activity that has an antidepressant effect.

55. The method of claim 54, wherein the optimal E_{brain} is approximately 110 Volts/meter to approximately 120 Volts/meter.

56. The method of claim 54, wherein E_{brain} is based on an E-field of the patient's whole brain.

57. The method of claim 54, wherein the finite element modeling comprises segmentation of the brain, extracranial electrode placement on the patient's scalp, tessellation of volume into a mesh, and determining the patient's baseline E_{brain}.

58. The method of claim 54, wherein the 90th percentile of E-field magnitudes ranges from approximately 0.1 to approximately 0.2 Volts/meter per milliampere of current.

59. The method of claim 54, wherein the method induces the antidepressant effect without cognitive impairment of the patient, wherein the cognitive impairment comprises one or more of amnesia, executive dysfunction, or verbal dysfluency.

60. The method of claim 54, wherein the optimal individualized current amplitude is applied to the patient's brain at a pulse width of approximately 0.3 to approximately 1.0 milliseconds.

61. The method of claim 54, wherein the optimal individualized current amplitude is applied to the patient's brain for a pulse train duration of approximately 0.5 to approximately 8 seconds.

62. The method of claim 54, wherein the optimal individualized current amplitude ranges from approximately 600 mA to approximately 1200 mA.

63. A method of determining an optimal individualized current amplitude for a patient for use in electroconvulsive therapy (ECT) to the patient's brain, comprising:
determining a baseline E-field strength (baseline E_{brain}) of the patient's brain by finite element modeling using structural MRI of the patient's brain with a fixed placement of extracranial electrodes, wherein the baseline E_{brain} comprises at least a 90th percentile of E-field magnitudes from all voxels in the patient's brain; and
dividing an optimal E-field strength (optimal E_{brain}) by the patient's baseline E_{brain} to produce the optimal individualized current amplitude for the patient, wherein the optimal E_{brain} is an E-field strength sufficient to induce an antidepressant effect without cognitive impairment.
